(19) United States
(12) Patent Application Publication
(10) Pub. No.: US 2025/0152625 A1
(43) Pub. Date: May 15, 2025

(54) CELLS AND METHODS

(71) Applicant: KING'S COLLEGE LONDON, London (GB)

(72) Inventors: Ashish Satish PATEL, London (GB); Bijan MODARAI, London (GB)

(21) Appl. No.: 18/838,329

(22) PCT Filed: Feb. 9, 2023
(86) PCT No.: PCT/GB2023/050299

§ 371 Date: Aug. 14, 2024

(30) Foreign Application Priority Data
Feb. 15, 2022 (GB) 2201997.0

(51) Int. Cl. A61K 35/15; A61P 9/10; C12N 5/0786

(52) U.S. Cl. CPC A61K 35/15; A61P 9/10; C12N 5/0645; C12N 2502/1382

(57) ABSTRACT

The invention relates to a population of cells, wherein said population of cells comprises at least 50% macrophage or monocyte cells, characterised in that at least 50% of said macrophage or monocyte cells express each of the markers: MRC1; TIE2; and CD163. The invention also relates to uses of those cells, methods of making them and methods of treating a subject by administering them.

### BACKGROUND TO THE INVENTION

Peripheral arterial disease affects 20% of individuals over 75. This can lead to critical limb ischaemia (CLI), which manifests as intractable pain, ulceration and gangrene. The quality of life in patients with CLI is similar to those with terminal cancer. 40,000-60,000 patients are diagnosed with CLI in the UK per year. ⅓ of these cannot be revascularised by conventional treatments such as bypass surgery/stenting and require an amputation. Estimated total of CLI patients worldwide approx. 237 million.

Therapeutic cell-based neovascularization has been heralded as a promising treatment for the salvage of critically ischaemic limbs with the aim of stimulating the growth of new blood vessels within the ischaemic tissue. However, numerous clinical trials of cell therapy have reported only modest benefits to date because ill-defined, heterogeneous populations of cells (mononuclear cells) from the bone marrow or peripheral blood have been used with little understanding of their activity (Fadini et al. “Autologous stem cell therapy for peripheral arterial disease meta-analysis and systematic review of the literature.” Atherosclerosis 2010 March 209(1); Qadura et al. 2018 Stem Cells February 36(2) “Cell Therapy for Critical Limb Ischemia: An Integrated Review of Preclinical and Clinical Studies.”).

It is widely accepted that an effective therapy is likely to require more specific, potent cell type(s), which can be isolated reliably and in sufficient numbers. These are problems in the art.

A small and specific subpopulation of mononuclear cells, monocytes/macrophages (Mo/MΦ), have been investigated and it is shown that those which express the marker TIE2 may regulate post-ischaemic revascularisation (Patel et al. 2013 “TIE2-expressing monocytes/macrophages regulate revascularization of the ischemic limb.” EMBO Mol Med 2013 May 7). Unfortunately, the numbers of these cells (˜3% of whole monocyte population) that can be isolated from the blood are too low for effective treatment.

Rybalko et al. 2017 (Regen. Med. Volume 12, number 2, pages 153-167) describe therapeutic potential of adipose-derived stem cells and macrophages for ischemic skeletal muscle repair. Rybalko et al. use a transwell approach in manipulating their cells. Rybalko et al. take in excess of 14 days to produce their cells. There is no disclosure of triple positive MRC1/TIE2/CD163 positive cells in Rybalko et al.

Kim and Hematti 2009 (“Mesenchymal stem cell-educated macrophages: a novel type of alternatively activated macrophages.” Exp Hematol December; 37 (12): 1445-53 (corresponding to published US patent application US2011/0045071 by Hematti and Kim)) disclose a method comprising first turning primary monocytes into macrophages by culturing for 7 days without cytokines (see page 1446—left-column—‘Cell Culture’ last paragraph), and the culturing those macrophages with MSCs. Kim and Hematti take in excess of 10 days to produce their cells. There is no disclosure of triple positive MRC1/TIE2/CD163 positive cells in Kim and Hematti.

The present invention seeks to overcome problem(s) associated with the prior art.

### SUMMARY OF THE INVENTION

The invention is concerned with cells which have been “educated” or “primed” or otherwise driven to express key markers MRC1, TIE2 and CD163. These cells exhibit advantageous functional properties for example in revascularisation. These cells therefore have direct industrial application for example in clinical settings where revascularisation is required, for example in peripheral vascular disease (such as critical limb ischemia (CLI)). Most suitably “educating” or “priming” refers to co-culture of the starting cells (monocytes/macrophages) with mesenchymal stem/stromal cells (MSCs), more suitably with mesenchymal stem cells. Suitably cells are human cells. Suitably the cells are in vitro or ex vivo human cells. Suitably the cells are not part of a human body. Suitably the cells are not germ line cells. Suitably the cells are not obtained by an essentially biological process, but rather are obtained involving in vitro manipulations. Suitably the cells are not from, suitably are not derived from, a human embryo. Suitably the cells are not totipotent cells.

In brief, these new cells are produced by starting with monocytes/macrophages from a subject, and manipulating them in vitro using carefully designed methods. Important steps in the method may include the ratio of monocytes/macrophages to stem cells in a co-culture step, and/or the length of time of the culture step(s), and/or the number of media changes used during this culture period. The inventors designed these methods thoughtfully on the basis of robust research and intellectual deliberation. The inventors discovered a surprising optimal window during which marker expression (and therefore cell function) can be stimulated. The remarkable surprising nature of these advances is demonstrated by the fact that continued culture over longer periods results in loss of the markers. Thus, the invention makes a new and surprising contribution to the art, enabling production of cells with new phenotypic characteristics, and with therapeutically beneficial functions.

The invention is based on these surprising advances.

Thus in one embodiment the invention provides a population of cells,
wherein said population of cells comprises at least 50% myeloid cells such as macrophage or monocyte cells,
characterised in that at least 50% of said myeloid cells such as macrophage or monocyte cells express each of the markers:
   MRC1;
   TIE2; and
   CD163.

Suitably said population of cells comprises at least 70% myeloid cells such as macrophage or monocyte cells.

Suitably said population of cells comprises at least 80% myeloid cells such as macrophage or monocyte cells.

In another embodiment the invention relates to a population of cells as described above wherein at least 60% of said myeloid cells such as macrophage or monocyte cells express each of the markers:
MRC1;
TIE2; and
CD163.

In another embodiment the invention relates to a population of cells as described above wherein said macrophage or monocyte cells express CD14 and/or CD45.

In another aspect the invention provides a population of cells,
wherein said population of cells comprises at least 50% CD14+ and/or CD45+ cells, characterised in that at least 50% of said CD14+ and/or CD45+ cells express each of the markers:
   MRC1;
   TIE2; and
   CD163.

Suitably said population of cells comprises at least 70% CD14+ and/or CD45+ cells.

Suitably said population of cells comprises at least 80% CD14+ and/or CD45+ cells.

In another embodiment the invention relates to a population of cells as described above wherein at least 60% of said CD14+ and/or CD45+ cells express each of the markers:
MRC1;
TIE2; and
CD163.

In another embodiment the invention relates to a population of cells as described above wherein said CD14+ and/or CD45+ cells are, or are derived from, myeloid cells. In another embodiment the invention relates to a population of cells as described above wherein said CD14+ and/or CD45+ cells are, or are derived from, circulating white blood cells.

In one embodiment suitably cells are in vitro cells or are ex-vivo cells.

Suitably the population of cells as described above comprises a 3:1 ratio of monocyte/macrophage cells (or CD14+ and/or CD45+ cells):MSCs.

Suitably the population of cells as described above comprises a 3:1 ratio of triple positive monocyte/macrophage cells (or triple positive CD14+ and/or CD45+ cells):MSCs.

Suitably the population of cells as described above comprises macrophage or monocyte cells expressing TNFalpha (TNFa) and/or IL-12. Suitably at least 60%, more suitably at least 80%, more suitably at least 90% of said CD14+ and/or CD45+ cells further express IL-12. Suitably at least 4%, more suitably at least 5%, more suitably more than 5% of said CD14+ and/or CD45+ cells further express TNFalpha (TNFa).

Suitably the population of cells as described above comprises cells wherein said cells are encapsulated.

In another embodiment the invention relates to a method comprising
(a) providing a macrophage or monocyte (or CD14+ and/or CD45+ cell) from a subject
(b) providing a MSC
(c) culturing said macrophage or monocyte (or CD14+ and/or CD45+ cell) with said MSC.

Suitably the method is a method of inducing expression of MRC1, TIE2 and CD163 in a macrophage or monocyte (or in a CD14+ and/or CD45+ cell).

Suitably the method is a method of producing a triple positive macrophage or monocyte (or a triple positive CD14+ and/or CD45+ cell).

Suitably step (c) comprises contacting said macrophage or monocyte (or CD14+ and/or CD45+ cell) with said MSC to produce a cell mixture, and culturing said cell mixture.

Thus suitably the invention relates to a method comprising
(a) providing a monocyte from a subject
(b) providing a MSC
(c) culturing said monocyte with said MSC,
wherein step (c) comprises contacting said monocyte with said MSC to produce a cell mixture, and culturing said cell mixture.

Suitably the ratio of (macrophages or monocytes) or (CD14+ and/or CD45+ cells):(MSCs) in step (c) is 3:1.

Suitably the cells are cultured for about 3 to 7 days.

Suitably the cells are cultured for about 3 to 5 days.

Suitably the cells are cultured for about 3 days.

The cells are suitably cultured for an interval to effectively induce triple-positive marker expression.

In one embodiment cells may be cultured for about 3 to 7 days.

In a more suitable embodiment, more suitably cells may be cultured for about 3 to 5 days, which has the advantage of producing more triple positive cells than culture for 7 days.

In a most suitable embodiment, most suitably cells may be cultured for about 3 days, which has the advantage of producing the greatest proportion of triple positive cells.

In one embodiment cells may be cultured for about 72 to 168 hours.

In a more suitable embodiment, more suitably cells may be cultured for about 72 to 120 hours, which has the advantage of producing more triple positive cells than culture for 168 hours.

In a most suitable embodiment, most suitably cells may be cultured for about 3 days (about 72 hours), which has the advantage of producing the greatest proportion of triple positive cells.

Suitably the cells are cultured in a medium, and wherein said medium is optionally changed every 3-5 days.

In one embodiment suitably said medium is changed every 3 days.

In one embodiment suitably said medium is changed every 4 days.

In one especially suitable embodiment, suitably said medium is changed every 5 days.

In one embodiment suitably said medium is changed every 7 days.

Most suitably if cells are cultured beyond 5 days, the medium is changed no later than day 5.

It is an advantage of the invention that the induction of triple marker expression is so efficient that culture of cells beyond day 5 is rarely required-therefore advantageously a change of medium is correspondingly rarely required, saving costs and saving labour, and avoiding risk of contamination at the time of medium change.

According to the methods of the invention, maximal/optimal triple positive marker expression is obtained at day 3 of culture. It is a further advantage of the invention that this optimal triple positive marker expression is maintained at day 4 and maintained at day 5.

It is also successfully observed that triple positive marker expression can continue at day 6 and can continue at day 7.

It is noted that triple positive marker expression can decline from day 7 so that cells cultured up until day 7 remain useful but perhaps a higher dose of such cells might be delivered to the patient so as to maintain efficacy.

Suitably cells are NOT cultured for an 8^{th} or further day. Suitably cells are NOT used if they have been cultured for an 8^{th} or further day.

Suitably cells are cultured to at least a 3^{rd} day. Suitably cells are NOT used until they have been cultured to at least day 3.

The invention also relates to a population of cells obtainable by a method as described above.

The invention also relates to a population of cells obtained by a method as described above.

In another embodiment the invention relates to a population of cells as described above for use in treatment of peripheral vascular disease, more suitably critical limb ischaemia (CLI), more suitably chronic limb threatening ischaemia (CLTI).

In another embodiment the invention relates to a population of cells as described above for use in treatment of fibrosis.

In another embodiment the invention relates to a population of cells as described above for use in treatment of ischaemic stroke.

Suitably treatment comprises administration of the population of cells to a subject.

Suitably treatment comprises administering a dose of about 10⁶-10⁹ said cells to a subject. Suitably treatment comprises administering said cells by injection.

In another embodiment the invention relates to a method of treating a mammalian subject comprising administering a population of cells as described above to said subject.

Suitably said method comprises administering a dose of about 10⁶-10⁹ said cells.

In another embodiment the invention relates to use of a population of cells as described above to induce angiogenesis in a mammal.

Also described is a composition comprising a population of cells as described above.

Suitably said composition is a pharmaceutical composition.

Also described is use of a composition as described above in medicine.

### DETAILED DESCRIPTION

One of the advances made by the inventors is to create an ex vivo method to generate large numbers of a specific population of monocytes/macrophages (Mo/M) expressing the marker TIE2 capable of stimulating the growth of new blood vessels for delivery into the ischaemic leg.

To do this, the inventors utilise the immunomodulatory effects of mesenchymal stem/stromal cells (MSCs). MSCs may be obtained from either the bone marrow (BM) or adipose tissue. MSCs modulate the properties of cells within their vicinity, including Mo/MΦs, by shifting their polarisation from an inflammatory to an anti-inflammatory phenotype (Kim and Hematti 2009 “Mesenchymal stem cell-educated macrophages: a novel type of alternatively macrophages.” activated Exp Hematol December; 37(12):1445-53 (corresponding to published US patent application US2011/0045071 by Hematti and Kim); Maggini et al. 2010 “Mouse bone marrow-derived mesenchymal stromal cells turn activated macrophages into a regulatory-like profile.” PLOS One 5(2):e9252.)

In Kim and Hematti 2009, they first turn primary monocytes into macrophages by culturing for 7 days without cytokines (see page 1446—left-column—‘Cell Culture’ last paragraph). Only then are the resulting macrophages exposed to MSCs. In contrast, the methods of the invention culture/contact primary monocytes directly with MSCs at Day 0. Thus, according to the present invention the myeloid cells such as primary monocytes are placed into culture immediately after isolation—the start time is the moment they are placed in culture. In the invention, primary monocytes are placed into culture with the MSCs, so there are no pre-differentiated macrophages in the mixture at this point. In prior art methods, first macrophage are generated and then (subsequently) those macrophage are contacted with MSCs. In a method different from the invention herein, Kim and Hematti 2009 first place their monocytes in culture for 7 days to differentiate them into macrophages, and then it is those macrophage cells (i.e. pre-differentiated macrophages) that are exposed to MSCs.

The inventors believe that the cells of Kim & Hematti 2009 are not cells as described herein i.e. are not triple expressers of MRC1 (CD206), TIES2 (CD202B) and CD163.

The reasons for this are firstly that the cells show differences in the expressed markers. This is because only a small proportion of the Kim & Hematti cells express even one of the markers (CD206), and this is typically as low CD206 expressers. For example, there is wide range of CD206 expression from 11.81% to 75.89% in Kim & Hematti (FIG. 3—A/C/E) which indicates an unreliable CD206 expression and a low average CD206 expression. (The average amount is 46.1%.) In contrast, our data show that cells of the invention are consistent (reliable) and high CD206 expressers with a mean CD206 expression of 92% (+/−SEM of 14%) (Re: 92%—we refer to FIGS. 2B and 11B. We also show that our method is reproducible with different technicians (skilled workers) and still delivers over 90% expression (see FIG. 4).

It is a benefit of the invention that monocytes/macrophages described herein expressing CD206 have a tissue remodelling phenotype and play an important role in the formation of new vessels (both angiogenesis and arteriogenesis). These cells also have an important anti-fibrotic function. This is demonstrated in the Examples section.

Secondly, the cells show different PROPERTIES. For example, the cells described herein show high TNFa (TNFalpha) levels, whereas those in Kim and Hematti 2009 (US2011/0045071) are low. TNFa is important in tissue remodelling. TNFa has a role in pathological angiogenesis and arteriogenesis. It stimulates collateral blood vessel growth in tissue after ischaemia. TNFa has also been shown to have a marked anti-fibrotic function and regulates the resolution phase of fibrosis.

The cells in Kim and Hematti have downregulated TNFa; Kim and Hematti disclose that the cells are downregulated by one-third compared with the control cells. In fact, their data (Kim and Hematti FIGS. 3E and F) show that only 3% of their cells express TNFa. The level of TNFa expressing cells drops to lower than the control cells whereas cells of the present invention show constant or elevated levels of TNFa expression so cells of the invention clearly do not downregulate TNFa as in Kim and Hematti.

We have quantified the amount expressed. The control cells express a median of 42 pg/mL, whereas our MSC-primed cells (cells of the invention) express high levels—a median 251 pg/mL—so we show that cells of the invention upregulate TNFa levels by 6-fold.

IL-12 is another potent cytokine that induces tissue remodelling. The cells of Kim & Hematti lose expression of IL-12, whereas in our cells IL-12 levels remain high. We refer to FIG. 27: Day 3: No difference in IL-12 expression (IL-12 is still highly expressed on our cells after co-culture, whereas the cells of Kim & Hematti do NOT express IL-12) Day 7: No difference in IL-12 expression (IL-12 is still highly expressed on our cells after co-culture, whereas the cells of Kim & Hematti do NOT express IL˜12 (e.g. approx. only 1.5% of cells in Kim and Hematti express IL-12).

The differences in these two cytokines alone show that the cells of the invention have very distinct properties from those of prior art such as Kim & Hematti. This is especially true as TNFa and IL-12 are inflammatory cytokines, expression of which is not expected.

It should be noted that unless otherwise apparent from the context, phrases high and low used to describe expression refer to the proportion of cells expressing rather than absolute levels of expression—e.g. proportions of cells deemed to be expressing by FACS analysis or similar binary counting method. This is apparent to the skilled worker because polypeptides such as TNFa/IL-12 are secreted so assessing levels of expression can be challenging to compare between different cells for example if they are grown in a larger volume of media then the apparent concentration of expressed protein per cell would be measured as lower (and vice-versa) and so expression levels are most often assessed as proportion of cells expressing unless otherwise noted.

We disclose techniques for culturing monocytes with MSCs in order to prime them (‘educate’ them) i.e. in order to switch them towards a different phenotype of pro-angio/arteriogenic monocytes/macrophages (Mo/MΦs).

In one embodiment ‘primed’ or ‘educated’ monocytes/macrophages means monocytes/macrophages co-cultured with MSCs, suitably monocytes/macrophages co-cultured with MSCs in vitro.

In addition the inventors disclose for the first time that ‘triple positive’ monocytes/macrophages (Mo/MΦs) expressing each of the markers
MRC1, and
TIE2, and
CD163;
   are extremely clinically useful as described herein.

Disclosed are conditions which enable the highest proportion of triple positive cells to be obtained from starting cells.

It is demonstrated that triple positive macrophage can be used to stimulate production of new blood vessels (revascularisation).

We disclose how to produce triple expression of the three key markers on macrophage.

It is disclosed how to generate triple expressing macrophage from mononuclear cells harvested from a patient's blood.

The inventors demonstrate the clear link of cells expressing the three markers disclosed herein to the functional benefit of stimulating new blood vessels.

The cells described herein are new by virtue of expressing the triple marker combination.

The sequences of the key markers are available to the skilled worker as they are publicly disclosed as shown in the table below:

| | Alternate | Accession | |
|---|---|---|---|
| Marker | Names | Number | Notes |
| MRC1 | CD206 | UniProt: | Macrophage mannose receptor 1 |
| | | P22897 | |
| TIE2 | VMCM, | UniProt: | Tyrosine-protein kinase that acts as |
| | VMCM1, | Q02763 | cell-surface receptor for angiopoietin |
| | TEK, | | 1, 2 and 4 and regulates angiogenesis, |
| | CD202B | | endothelial cell survival, proliferation, |
| | | | migration, adhesion and cell spreading, |
| | | | as well as vascular quiescence. |
| CD163 | M130 | UniProt: | Scavenger receptor cysteine-rich type 1 |
| | | Q86VB7 | protein M130 (Haemoglobin scavenger |
| | | | receptor) |

| Mo/MΦ | Alternate | Accession | |
|---|---|---|---|
| Marker | Names | Number | Notes |
| CD45 | L-CA, | UniProt: | Leukocyte common antigen |
| | T200, | P08575 | |
| | PTPRC | | |
| CD14 | CD_antigen: | UniProt: | Monocyte differentiation antigen |
| | CD14 | P08571 | CD14, cluster of differentiation 14. |
| | | | Myeloid cell-specific leucine-rich |
| | | | glycoprotein. LPS receptor subunit. |
| CD16 | Fc-gamma | UniProt: | |
| | receptor IIIb | Q9ULV2 | |
| | (CD16) | | |

### Threshold for Expression:

Suitably ‘expressing’ the markers means that the protein can be detected by immunostaining such as antibody staining e.g. flow cytometry.

Unless otherwise apparent, expression refers to expression of the full length protein, or to expression of the biologically relevant part e.g. the biologically active part. In one embodiment expression refers to expression of the marker at the cell surface. In one embodiment, when the marker is an external/cell surface protein, expression refers to expression of the marker sufficient to enable detection at the external cell surface.

In case any further guidance is needed, normal macrophages or monocytes produce proteins such as CD14 and/or CD45; a cell is considered a monocyte or macrophage if it ‘expresses’ CD14 and/or CD45 as detected by FACS using an anti-CD14 and/or anti-CD45 reagent such as an anti-CD14 and/or anti-CD45 antibody, or anti-CD14 and/or anti-CD45 affimer. A cell expressing CD14 or CD45 is designated CD14+ or CD45+ as is conventional in the art. If only one of CD14/CD45 is used, suitably only CD45 is used which has the advantage of superior technical performance/ease.

For markers not normally expressed on macrophages or monocytes, but whose expression is induced by the invention (e.g. MRC1, TIE2, CD163), a cell is considered to ‘express’ the marker according to the present invention (i.e. considered positive for the marker) when the flow cytometer detects a threshold amount of fluorescence that is higher relative to the same cells that do not express these markers (i.e. the negative controls). It is routine for a person skilled in the art to select appropriate fluorescence threshold value(s).

In case any further guidance is needed, a suitable negative control would be a sample of the macrophages or monocytes which have NOT been co-cultured with MSCs (mesenchymal stem/stromal cells) as taught herein. For example, a negative control may be a population of unprimed/untreated macrophages or monocytes isolated from peripheral blood.

More suitably a negative control may be a population of primary monocytes isolated from peripheral blood. Suitably the negative control cells have not been cultured with MSCs. Suitably the negative control cells have not been cultured in vitro at all (primary monocytes). Monocytes isolated from blood do not triply express the markers of the invention so are a very useful negative control.

Macrophage expressing these three markers in combination have never been described before to the best of the inventors' knowledge.

Suitably the term ‘about’ applied to a numeric value means +/−1% of the stated value.

Suitably the term mesenchymal stem cell has its natural meaning in the art. The abbreviation “MSC” means mesenchymal stem/stromal cells (MSCs), more suitably means mesenchymal stem cell.

### Monocytes (Mo) and Macrophages (MΦs)

Monocytes (Mo) are defined as blood mononuclear cells with bean-shaped nuclei that express CD11b, and CD14 (LPS receptor subunit) in humans. Non-classical monocytes may express CD16 in addition to lower levels of CD14, and intermediate monocytes express CD14 and CD16. Monocytes (Mo) differentiate into macrophages (MΦs).

CD14 is regarded as a unique marker of Monocytes (Mo), i.e. if CD14 is found on a circulatory cell, it is regarded as a Monocyte (Mo). Monocytes (Mo) also display other markers such as CD45, CD11b (or both).

Macrophages are a diverse group of white blood cells known for eliminating pathogens through phagocytosis. Human macrophages typically express CD14, CD40, CD11b, CD64, EMR1, lysozyme M, MAC-1/MAC-3, 25F9 and CD68.

The cells of the invention also express others markers including HLA-DR and CD38 (see FIG. 7).

In the past, macrophages have been classified according to the organ in which they were found (e.g. Kuppfer cells in the liver, Langerhans cells in the skin, etc). However, the current nomenclature has shifted away from organ-specific naming of macrophages to “M1” and “M2” macrophages. This classification is based upon macrophage polarisation rather than macrophage location.

M1 macrophages are classically activated, typically by IFN-γ or lipopolysaccharide (LPS), and produce proinflammatory cytokines, phagocytise microbes, and initiate an immune response. M1 macrophages produce nitric oxide (NO) or reactive oxygen intermediates (ROI) to protect against bacteria and viruses.

M2 macrophages are alternatively activated by exposure to certain cytokines such as IL-4, IL-10, or IL-13. M2 macrophages will produce either polyamines to induce proliferation or proline to modulate collagen production. These macrophages are associated with wound healing and tissue repair. There are three types of M2 macrophages: M2a, M2b, and M2c. M2 macrophages also contribute to the formation of extracellular matrix and do not produce nitric oxide or present antigen to T cells. Tumor-infiltrating macrophages are typically classified as M2, although some classify them as myeloid-derived suppressor cells (MDSC).

There are conflicting views in the art regarding which subset of macrophages are the most effective. In contrast, the inventors' approach is not to generate a specific subset that fit within either a ‘monocyte’ or an ‘Mt’ or ‘M2’ macrophage category, but to produce macrophages that express 3 specific markers (TIE2, CD163 and MRC1). Thus, the discussion in this document of the cells of the invention often mentions ‘monocytes’ and ‘macrophages’ together (for example “Mo/MΦs”).

In a strict classification, the circulating white blood cells which are isolated and then co-cultured with MSCs as described herein can be viewed as pure monocytes because they are circulating whereas macrophages are usually considered tissue cells. However, the invention is not concerned with theoretical questions such as (for example) whether the starting cells have differentiated in culture into macrophages. It can be observed that they are beginning to, but they still have monocytic properties (CD14 expression). Indeed, reduced expression of MMP-9 (which is a matrix metalloproteinase that is increased when monocytes start to differentiate into macrophages) can be seen compared with the monocytes that are cultured alone (FIG. 18). Without wishing to be bound by theory, it can be said that they are “myeloid” cells as this encompasses both Mo/MΦs. For the purposes of the invention the starting cells (i.e. the blood mononuclear cells) may comprise monocytes or macrophages or a mixture of monocytes and macrophages.

More suitably, the starting cells (i.e. the blood mononuclear cells) may comprise monocytes such as primary monocytes.

A primary monocyte is defined as a CD14-expressing monocyte that has just been isolated from the blood and not yet been placed in culture (i.e. it is still in suspension). As soon as the monocyte is placed in a culture dish, it will stick (adhere) and is no longer a primary monocyte as it will start to slowly differentiate into a macrophage. In vitro incubated monocytes were primary monocytes prior to culture.

Suitably cells of the invention are co-cultured with MSCs for 100% of their culture time i.e. the starting cells (monocytes such as primary monocytes in suspension) are mixed with MSCs and placed into culture at day 0 and are co-cultured with MSCs for their entire time in culture. Prior art methods typically first prepare macrophages by culturing monocytes such as primary monocytes for 7 days and only then contacting those macrophage with MSCs. Suitably the cells of the invention are or have been co-cultured with MSCs for 100% of their culture time. Prior art methods such as Kim & Hematti co-culture their macrophages with MSCs for only 3-4 days out of 10-11 total days of culture. So at most Kim & Hematti co-culture their macrophages with MSCs for about 30% (3/10 d) to 36% (4/11 d) of total culture time.

In the unlikely event of further guidance being needed, suitably cells of the invention are derived from circulating white blood cells.

Suitably cells of the invention are myeloid cells.

Suitably cells of the invention are CD14+.

Suitably cells of the invention are CD45+.

Most suitably cells of the invention are CD14+ and CD45+.

Most importantly, the cells of the invention are non-naturally-occurring ‘triple positive’ cells. Since these cells are non-naturally-occurring it is not always helpful to classify them as ‘monocyte’ or ‘macrophage’, hence they are typically referred to collectively as “Mo/MΦs”. In one embodiment the cell of the invention is a triple-positive monocyte. In one embodiment the cell of the invention is a triple-positive macrophage.

Macrophage “polarization” for tissue repair/remodelling has been mentioned in the prior art. However, the macrophages reported in the prior art express one or more ‘M2’ markers. Suitably the cells of the invention do not exclusively express M2 markers. Suitably the cells of the invention are not exclusively M1 macrophage. Suitably the cells in the invention are not exclusively M2 macrophage. Suitably the cells of the invention are not exclusively M2a macrophage. Suitable the cells in the invention are not exclusively M2b macrophage. Suitably the cells of the invention are not exclusively M2c macrophage.

Suitably the cells of the invention are, or are derived from, blood mononuclear cells.

Suitably the cells of the invention are human cells.

Suitably the cells of the invention are CD45+ (i.e. suitably the cells of the invention express the CD45 marker).

Most suitably the cells of the invention are CD14+ (i.e. suitably the cells of the invention express the CD14 marker).

Suitably the cells of the invention are myeloid cells.

Suitably the cells of the invention are monocytes or macrophages.

Suitably the cells of the invention are monocytes.

Suitably the cells of the invention are macrophages.

We teach a method to generate CD45+Mo/Mos that express TIE2, MRC1 and CD163 in combination. These cells are able robustly to promote blood vessels growth within ischaemic tissue. These cells are very rare in the circulation (<1% of monocytes) and so the invention has the advantage of allowing for the production of up to 1-2 billion of these cells in a single cycle/from a single starting pool of Mo/Mos from leukapheresis by educating whole population(s) of monocytes with MSCs. A further advantage of this approach is that these cells can be produced and then cryopreserved (e.g. in batches) so that a patient can have a prolonged course of autologous therapy with multiple injections over the course of several weeks/months. This is not possible with the limited number of CD45+Mo/MΦs that may occur naturally. The autologous nature of the therapy reduced or removes the risks to the patient of adverse immunological reactions.

Although the generation of ‘M2’ macrophages has been disclosed, the known methods require prolonged culture using a combination of growth factors, which is a problem in the art. By contrast, the invention obviates the need for growth factors and provides the ability to generate these cells with this specific phenotype and function within just 5 days. This allows for the rapid treatment of patients who often present as acute-on-chronic emergencies.

Thus, in contrast to prior art approaches such as co-delivery of murine adipose-derived stem cells and macrophages to stimulate angiogenesis in the ischaemic murine limb, our approach uses MSCs as a way to prime monocytes. The MSCs are used only in the process of creating the ‘triple-positive’ monocytes/macrophages and are not part of the final therapeutic product. In one embodiment suitably some residual MSCs may remain in the cell population(s) administered. More suitably the MSCs are removed so that the administered cells consist essentially of, or consist of, triple positive macrophage cells and/or triple positive monocyte cells.

### Co-Culture

To the best of the inventors' knowledge, there is no known prior disclosure that the “triple-expressing” (TIE2, MRC1 and CD163) monocytes of the invention can be generated by co-culture using MSCs. Furthermore, the inventors also determined and disclose herein the optimal conditions to generate this potent population of cells.

To the best of the inventors' knowledge, no data exist in the literature on how these cells can be generated from whole population monocytes, or even that their generation is possible.

The inventors tested a range of 4 different time points as well as a range of 4 different ratios of monocytes: MSCs to determine the conditions that are successful in order to generate these cells (See Example 5).

Before the invention it was also unknown whether the monocytes from patients with peripheral arterial disease (such as CLI) could even be primed in this way.

The teachings and experimental data herein indicate that the immunogenic properties exerted by MSCs are not limited just to educating monocytes from healthy adults. It is further demonstrated that it is possible to use monocytes from aged patients with multiple co-morbidities to generate the cells/cell populations of the invention for autologous cell therapy.

### Co-Culture Conditions

Suitably isolated monocytes/macrophages, and/or cell mixtures of monocytes/macrophages and MSCs, are cultured in monocyte media.

Exemplary monocyte media comprises: RPMI-1640 (‘Roswell Park Memorial Institute’-1640) supplemented with 10% foetal calf serum (FCS) and optionally 1% anti-biotic/-mycotic.

Suitably cells are cultured in a humidified incubator.

Suitably cells are cultured at 37° C.

Suitably cells are cultured at 5% CO₂.

Suitably RPMI-1640 may be from any source, such as from ThermoFisher Scientific (Fisher Scientific-UK Ltd, Bishop Meadow Road Loughborough, United Kingdom, LE11 5RG) Catalogue number: 11875101.

In embodiments directed at production of clinical grade material (e.g. products intended for administration to human or animal subjects, suitably human subjects, suitably X-VIVO™-10, Serum free hematopoietic cell medium, with L-Glutamine, gentamicin and phenol red, xenofree is used instead of the RPMI medium. Suitably X-VIVO™-10 is from Lonza Group Ltd, Muenchensteinerstrasse 38, CH-4002 Basel, Switzerland, Catalog #: BE04-380Q.

Alternatives to X-VIVO™-10 media include TexMACS buffer which needs to be supplemented with either GMP grade 5% human serum albumin (Catalogue No. 623160054 from Biotest U.K. Ltd, 17 High Street, Longbridge, Birmingham, B31 2UQ, United Kingdom) or 5% human Serum Albumin (Albunorm™ Catalogue No. P/L 10673/0031 from Octapharma Ltd, The Zenith Building, 26 Spring Gardens, Manchester M2 1AB, U.K.).

Further details may be found in the examples section.

The cells of the invention do not occur naturally. The cells are only produced by human intervention using the methods described herein.

It is possible that a de minimis/negligible number of triple-positive cells might exist in the blood, but at very low (<1%) numbers, where they are almost undetectable. Even if such cells might be shown to exist, it will be noted that they do NOT express the 25F9 marker, which suitably the cells of the invention do express.

Suitably the cell(s) of the invention are ex-vivo.

Suitably the cell(s) of the invention are in vitro.

Suitably the cell(s) of the invention are isolated. Suitably isolated means removed from or separated from at least one component of their natural environment. For example isolated may mean removed from a human or animal body. For example isolated may mean separated from red blood cells (erythrocytes).

Suitably the cell(s) of the invention are present in a population of cells.

Suitably the invention provides a population of cells.

Suitably said population of cells comprises 70% or more proportion of macrophages or monocytes.

Suitably said population of cells comprises 0% proportion of erythrocytes.

Suitably said population of cells comprises 30% or less proportion of MSCs.

These are the release criteria of the final product that as in the clinical trial.

Cells which are singly positive for one of the triple markers disclosed herein may have been observed in the prior art. However, the combination of triple positive markers has never been observed on cells such as macrophage before. The inventors force this specific phenotype into the cells using the methods described herein.

Without wishing to be bound by theory, it may be that MRC1/TIE2 double positive cells may be useful in treating patients as described herein. Thus in one specific embodiment is described a macrophage or monocyte expressing each of the markers MRC1 and TIE2. Thus in one specific embodiment is described treatment of patients using said double positive cells. Thus in one specific embodiment is described said double positive cells for use in treatment of disease(s) disclosed herein, for example CLI. However, most suitably the cells used in the invention are triple positive for MRC1, TIE2 and CD163.

Rybalko et al. 2017 (Regen. Med. Volume 12, number 2, pages 153-167) describe therapeutic potential of adipose-derived stem cells and macrophages for ischemic skeletal muscle repair. The methods used in Rybalko et al. would not produce triple expression of MRC1, TIE2 and CD163, or at least would not produce triple expression of MRC1, TIE2 and CD163 in any clinically useful amount. Rybalko et al. use a transwell approach in manipulating their cells. The inventors assert that this approach does not produce cells according to the present invention. Rybalko et al. take in excess of 14 days to produce their cells. There is no disclosure of triple positive MRC1/TIE2/CD163 positive cells in Rybalko et al.

In more detail, Rybalko et al. does not disclose cell compositions/cell populations of the present invention. It is possible that a tiny number/tiny proportion of triple positive cells as described herein exist amongst the cell population created by following the teachings of Rybalko et al. However, firstly even if the triple positive cells exist in Rybalko et al. cell populations, they exist only at a “de minimis” level. For example, the inventors assert that no more than approximately 0.3% of cells in Rybalko at al. would be triple positive. Even by practicing the methods of Rybalko et al. with the knowledge of the invention that the triple positive cells are clinically useful, the best that can be obtained even using this hindsight knowledge to try to maximise the number of triple positive cells available from the Rybalko method is approximately 2% triple positive cells amongst the Rybalko et al. cell population. In contrast, the present inventors teach methods for generating populations of cells having at least about 50% triple positive cells. The maximum percentage of triple positive cells generated in Rybalko et al. is approximately 1-2%, which equates to the number the triple positive cells which would be generated if no treatment was applied e.g. if the cells were merely cultured. Suitably the cell composition of the invention comprises at least about 50% triple positive cells. In case any further evidence is needed, we refer to the Examples section which present comparative data.

### Dose

Suitably the invention provides a dose of cells.

In one embodiment suitably a dose of cells comprises approximately 10e6 to 10e9 cells (i.e. 10⁶-10⁹ cells, sometimes written 10\hat{ }6-10\hat{ }9 cells). More suitably a dose comprises 10⁶-10⁹ monocytes. In one embodiment a dose consists of 10⁶-10⁹ monocytes. In one embodiment suitably a dose of cells comprises approximately 10⁶-10⁹ cells, of which cells at least 50% are monocytes/macrophages, more suitably least 60% are monocytes/macrophages. In one embodiment a dose of cells comprises approximately 10⁶-10⁹ cells, within which at least 50% of the monocytes/macrophages present are triple positive monocytes/macrophages.

A suitable dose according to the invention comprises about one hundred million monocytes/macrophages per injection.

In one embodiment suitably one dose is administered to the subject each week.

In one embodiment suitably a course of treatment is ten weeks i.e. ten doses at one dose per week.

Most suitably, in one embodiment suitably a dose of cells comprises approximately 1×10e8 to 2×10e8 cells (i.e. 1×10⁸-2×10⁸ cells, sometimes written 1×10\hat{ }8-2×10\hat{ }8 cells) i.e. 100 million to 200 million cells per dose. More suitably a dose comprises 100 million to 200 million monocytes/macrophages of the invention. In one embodiment a dose consists of 100 million to 200 million monocytes/macrophages of the invention.

In one embodiment suitably a dose of cells comprises approximately 100 million to 200 million cells, of which cells at least 50% are monocytes/macrophages, more suitably least 60% are monocytes/macrophages. In one embodiment a dose of cells comprises approximately 100 million to 200 million cells, within which at least 50% of the monocytes/macrophages present are triple positive monocytes/macrophages.

In one embodiment suitably one dose is administered to the subject every 6-12 weeks, most suitably every 12 weeks or 3 months.

In one embodiment suitably a course of treatment is 3-6 doses.

In one embodiment suitably a course of treatment is 5 doses or 200 million cells per dose.

In one embodiment suitably a dose is 2-3 million cells per Kg of weight of subject. Thus, for an adult male weighing 75 Kg a dose is suitably 150 million to 225 million cells, more suitably 150 million to 200 million cells. It is routine for a clinician to calculate doses for other weights or sexes given this information.

A dose may comprise more than one hundred million cells, for example there may be a low level of stem cells from the co-culturing of the macrophage of the invention present in a dose to be administered. For example, a dose may comprise additional cells up to approximately 10% of the number of macrophage of the invention contained in the dose. For example, if the dose comprises one hundred million macrophage of the invention, the total number of cells in the dose may for example reach one hundred and ten million-one hundred million macrophage according to the invention plus an additional ten million “other” cells, for example stem cells originating from the co-culture step in production of the macrophage of the invention. In this example the dose comprises 90.9% macrophage of the invention (100 m macrophage of the invention/110 m total cells=90.9%).

Suitably the dose comprises at least 50% cells of the invention; more suitably at least 60%; more suitably at least 65%; more suitably at least 69%; more suitably at least 70%; more suitably at least 80%; more suitably at least 85%; more suitably at least 90%; more suitably at least 95%; more suitably at least 96%; more suitably at least 97%; more suitably at least 98%; most suitably at least 99% or more cells according to the invention. In a preferred embodiment, suitably the dose comprises at least 98%, or at least 99% or more, cells according to the invention such as triple positive macrophage expressing MRC1, TIE2 and CD163.

In one embodiment cell numbers of doses discussed herein refer to the numbers of triple positive monocyte/macrophage cells of the invention.

Regarding cell preparations for dose manufacture, in a typical method of the invention 2 billion whole population monocytes are obtained from leukapheresis and used as starting cells in the method of the invention, which means up to about 1-1.4 billion will be triple positive at the end of co-culture. This allows for 1 or 2 extra-large doses of over a billion cells or more suitably allows for multiple doses (e.g. 6 doses) of up to 1-200 million cells/dose.

Without wishing to be bound by theory, based on the inventors' 3% triple positive finding for patients with CLI, any of the prior art noted herein, who use day 7, will generate 60 million cells—not even useful for a single dose.

### Cell Numbers

Percentage of cells in relation to cell type is suitably the percentage of the whole population of cells which is the specified cell type. Therefore a figure of “at least 25% macrophage or monocyte cells” suitably means 25% of the cells in the whole population of cells are macrophage or monocyte cells. For example if there are 1000 cells in a sample, “25% macrophage or monocyte cells” means 25% of the cells within the whole population of 1000 cells are macrophage or monocyte cells (i.e. 250/1000 are macrophage or monocyte cells).

Percentage of cells in relation to marker expression is suitably the percentage of the monocyte cell population expressing the recited markers. Therefore, a figure of 50% “triple positive cells” suitably means 50% of the monocytes in a cell population are triple positive. For example if there are 1000 cells in a sample, 50% “triple positive cells” means 50% of the monocytes within the 1000 cells are triple positive. So if there are 100 monocytes within the total number of cells of 1000 cells, 50% “triple positive cells” means 50 triple positive monocytes (50/100 monocytes triple positive) out of the overall cell count of 1000. Suitably the cells of the invention comprise at least 50% “triple positive cells”, i.e. suitably 50%, or more suitably greater than 50%, triple positive cells (monocytes). It must be noted that references to ‘monocytes’ here may be replaced with references to ‘monocytes/macrophages’ or ‘Mo/MΦ’ or ‘CD14+ and/or CD45+ cells’ such as ‘CD14+ and/or CD45+ myeloid cells’ or ‘mononuclear cells harvested from the subject's blood’ as is apparent from the rest of this document. Discussion in relation to monocytes is shorthand in order to aid understanding and is not intended to exclude or omit the other cell types noted.

Suitably the proportion of the population of cells produced according to the method of the invention which are ‘monocytes/macrophages’ (or ‘Mo/MΦ’ or ‘CD14+ and/or CD45+ cells’ such as ‘CD14+ and/or CD45+ myeloid cells’ or ‘mononuclear cells harvested from the subject's blood’) is at least 50%, more suitably at least 60%, more suitably at least 70%, more suitably at least 80%; more suitably at least 90%; more suitably at least 95%; more suitably at least 96%; more suitably at least 97%; more suitably at least 98%; most suitably at least 99% or more cells according to the invention. In other words, if the method produces a population of mononuclear cells harvested from the subject's blood co-cultured with MSCs then after the co-culture is complete (and after optional purification/enrichment of the mononuclear cells after co-culture if desired), suitably at least 50% of the cells present in the population are ‘monocytes/macrophages’ (or ‘Mo/MΦ’ or ‘CD14+ and/or CD45+ cells’ such as ‘CD14+ and/or CD45+ myeloid cells’ or ‘mononuclear cells harvested from the subject's blood’).

Suitably the proportion of cells produced according to the method of the invention expressing the triple positive markers is at least 30%, more suitably at least 40%, more suitably at least 50%, more suitably at least 60%; more suitably at least 65%; more suitably at least 69%; more suitably at least 70%; more suitably at least 80%, more suitably at least 85%; more suitably at least 90%; more suitably at least 95%; more suitably at least 96%; more suitably at least 97%; more suitably at least 98%; most suitably at least 99% or more cells according to the invention. In other words, if the method is started with an input of one billion mononuclear cells harvested from the subject's blood, most suitably the method of the invention converts at least (e.g.) approximately 80% of those mononuclear cells to triple expressing mononuclear cells i.e. eight hundred million triple expressing cells from an input of one billion mononuclear cells.

Suitably said mononuclear cells comprise macrophages or monocytes.

In one embodiment suitably said mononuclear cells comprise monocytes.

More suitably in one embodiment said mononuclear cells comprise macrophages.

In a most suitable embodiment the invention relates to a cell population comprising at least 70% macrophage or monocyte cells (or ‘Mo/MP’ or ‘CD14+ and/or CD45+ cells’ such as ‘CD14+ and/or CD45+ myeloid cells’ or ‘mononuclear cells harvested from the subject's blood’), and at least 50% of said macrophage or monocyte cells express each of the markers:
MRC1;
TIE2; and
CD163,

### Triple Positive

Suitably cells are considered to express the marker(s) discussed herein when said marker(s) are detectable for example using immunodetection to assay expression.

Suitably when cells are described as “triple positive”, this means that those cells are positive for MRC1, TIE2 and CD163.

A cell is considered “triple positive” when each of those markers is detectable as described above.

It is an advance of the invention which teaches that the triple positive cells are clinically useful. Although it may be that a tiny fraction of a prior art cell population might comprise triple positive cells as described herein, there has never been any teaching in the art that those triple positive cells are clinically useful. Thus, in one embodiment the invention relates to use of a triple positive cell as described herein in medicine.

### Additional Marker—25F9

Suitably cells of the invention express human 25f9, a marker which is not present on circulating monocytes. 25F9 is a marker showing that a monocyte is maturing into a macrophage.

Cells begin to express 25F9 in culture as described above.

Cells express 25F9 at day 3 in culture as described above. This shows that cells of the invention mature very quickly in the presence of the MSCs.

These cells (triple positive cells also expressing 25F9) do not circulate naturally in the blood. This advantageous expression pattern is produced only by manipulation as taught herein e.g. ex vivo manipulation such as co-culture with MSCs.

25F9 may be detected by any suitable method known in the art.

Most suitably detection of 25F9 is by use of affinity reagent such as an antibody (anti-25F9 antibodies) capable of specifically binding 25F9 protein at the cell surface to detect for cellular expression. Suitably anti-25F9 antibody may be from any source.

Most suitably detection of 25F9 is by use of the following antibody (Mature Macrophage Marker Monoclonal Antibody (eBio25F9 (25F9)), eFluor 660, eBioscience): catalogue number 50-0115-42 from Thermo Fisher (Fisher Scientific-UK Ltd, Bishop Meadow Road Loughborough, United Kingdom, LE11 5RG). Suitably the detection is carried out in accordance with manufacturer's instructions.

Suitably said antibody is Mouse/IgG1, kappa. Recommended isotype control is Mouse IgG1 kappa Isotype Control (P3.6.2.8.1), eFluor 660, eBioscience. Suitably antibody is stored in PBS, pH 7.2, with 0.1% gelatin, 0.2% BSA. Optional 0.09% sodium azide. Do not freeze. Store in dark at 4 Celsius.

The monoclonal antibody 25F9 recognizes a protein on mature macrophages both on the cell surface and in intracellular vesicular structures. Expression is not found on immature macrophages or monocytes or any other hematopoietic cell. Applications Reported: This eBio25F9 (25F9) antibody has been reported for use in flow cytometric analysis. Applications Tested: This eBio25F9 (25F9) antibody has been pre-titrated and tested by flow cytometric analysis of cultured human macrophages from monocytes. This can be used at 5 μL (0.25 μg) per test. A test is defined as the amount (μg) of antibody that will stain a cell sample in a final volume of 100 μL. Cell number should be determined empirically but can range from 10\hat{ }5 to 10\hat{ }8 cells/test. eFluor® 660 is a replacement for Alexa Fluor® 647. eFluor® 660 emits at 659 nm and is excited with the red laser (633 nm). Please make sure that your instrument is capable of detecting this fluorochome. Excitation: 633-647 nm; Emission: 668 nm; Laser: Red Laser. Filtration: 0.2 μm post-manufacturing filtered.

Suitably at least 50% of the monocytes/macrophage cells of the invention are triple positive and express 25F9. We typically observe at least 80% of the cells of the invention expressing 25F9. Thus, with reference to FIG. 11B, 80% of 69.96% triple positive cells=55.968% (triple positive+25F9) of cells in that preparation are (triple positive+25F9). We refer to FIG. 24 which shows that there is a significant increase in expression of 25F9 in MSC-primed monocytes compared with monocytes cultured alone.

### Medical Indications

The invention finds application in peripheral vascular disease.

The invention finds application in peripheral arterial disease.

The invention finds application in critical limb ischemia.

The invention finds application in fibrosis.

In one embodiment the invention finds application in treatment of fibrosis such as lung fibrosis. In one embodiment suitably the subject has, or has previously had, COVID-19.

This is especially helpful as COVID-19 patients can acquire long term lung problems.

The invention finds application in ischaemic stroke.

In one embodiment suitably the subject has asymptomatic Peripheral Arterial Disease (PAD). Thus the invention may be for use in treatment of Peripheral Arterial Disease (PAD). In one embodiment the invention provides a method of treatment of Peripheral Arterial Disease (PAD) comprising administering the cells and/or compositions of the invention to a subject.

In one embodiment suitably the subject has Intermittent Claudication. Thus the invention may be for use in treatment of Intermittent Claudication. In one embodiment the invention provides a method of treatment of Intermittent Claudication comprising administering the cells and/or compositions of the invention to a subject.

In one embodiment suitably the subject has Deteriorating Claudication. Thus the invention may be for use in treatment of Deteriorating Claudication. In one embodiment the invention provides a method of treatment of Deteriorating Claudication comprising administering the cells and/or compositions of the invention to a subject.

The cells and/or compositions of the invention may be for use in treatment of critical limb ischemia or critical limb threatening ischemia (CLTI). In one embodiment the invention provides a method of treatment of critical limb ischemia or critical limb threatening ischemia (CLTI) comprising administering the cells and/or compositions of the invention to a subject.

The cells and/or compositions of the invention may be for use in treatment of fibrosis.

The cells and/or compositions of the invention may be for use in treatment of fibrosis such as lung fibrosis. In one embodiment the invention provides a method of treatment of fibrosis comprising administering the cells and/or compositions of the invention to a subject. In one embodiment the invention provides a method of treatment of fibrosis comprising administering the cells and/or compositions of the invention to a subject wherein said subject has, or has previously had, COVID-19, e.g. for use in treating COVID-19 induced lung fibrosis.

The cells and/or compositions of the invention may be for use in treatment of other forms of interstitial lung disease, such as idiopathic pulmonary fibrosis.

The cells and/or compositions of the invention may be for use in treatment of stroke, suitably ischaemic stroke. In one embodiment the invention provides a method of treatment of stroke comprising administering the cells and/or compositions of the invention to a subject.

The cells and/or compositions of the invention may be for use in post-ischemic revascularisation. More suitably post-ischemic revascularisation of the heart, brain or leg. In one embodiment the invention provides a method of induction of post-ischemic revascularisation comprising administering the cells and/or compositions of the invention to a subject.

The cells and/or compositions of the invention may be for use in induction of growth of blood vessels e.g. angiogenesis and/or arteriogenesis. In one embodiment the invention provides a method of induction of angiogenesis and/or arteriogenesis comprising administering the cells and/or compositions of the invention to a subject.

The cells and/or compositions of the invention may be for use in stimulating production of new blood vessels in a mammal.

The cells and/or compositions of the invention may be for use in stabilising blood vessels. In one embodiment the invention provides a method of stabilising blood vessels comprising administering the cells and/or compositions of the invention to a subject.

The cells and/or compositions described herein are useful in treating fibrosis. Thus the composition of the invention may be used to treat fibrosis.

Fibrosis may be fibrosis of the kidney, fibrosis of the liver, fibrosis of the heart and/or fibrosis of the lung.

The inventors disclose that the triple positive cells described herein produce hepatocyte growth factor (HGF) and/or IL-10 and TNFα. These are antifibrotic proteins. This is an additional benefit of the cell compositions of the invention. The cells of the invention also respond to an inflammatory stimulus with lipopolysaccharide (LPS) to produce higher levels of IL-10 and TNFα, but not HGF. We refer to the figures.

Suitably administration is intramuscularly e.g. into the ischaemic limbs of patients with peripheral vascular disease such as peripheral arterial disease (e.g. CLI).

The invention may be used as an adjunctive treatment in a subject having revascularisation of their proximal inflow vessels.

The invention may be used as a stand-alone treatment for a subject who is a poor surgical candidate.

The invention may also be used intra-operatively in patients having endovascular revascularisation procedures, where the primed cells could be delivered directly to the end arterioles at the sites of known vessel occlusions.

The invention may also be used for the treatment of other cardiovascular conditions where tissue ischaemia is present, including patients with ischaemic heart disease, heart failure and/or those with cerebrovascular disease (e.g. stroke).

### Cell Production

In one embodiment the cells/cell populations of the invention may be produced by providing whole population monocytes isolated from patients (suitably previously isolated i.e. in one embodiment suitably the step of providing a population of monocytes comprises providing an in vitro population of monocytes).

Suitably monocytes are obtained by leukapheresis. This has the advantage of providing a high number of cells.

Suitably monocytes are obtained by single blood donation (e.g. up to 485 ml). This has the advantage of not requiring leukapheresis machinery.

Suitably the monocytes/macrophage are human monocytes/macrophage.

### Methods to Isolate Monocytes/Macrophage

Monocytes/macrophage can be isolated according to their expression of CD14. These cells can be either
labelled with immunomagnetic anti-CD14 beads and isolated by passing them through a magnetic column, or
fluorescently stained with CD14 and then extracted using fluorescence-activated cell sorting.

Suitably the monocytes are then primed with MSCs.

Suitably the MSCs are human MSCs.

MSCs may be from any suitable source e.g. from either the bone marrow or from adipose tissue (suitably previously isolated i.e. in one embodiment suitably the step of providing MSCs comprises providing an in vitro population of MSCs).

MSCs from a range of sources may be used in the invention, for example commercially available MSCs and/or GMP-ready MSCs may be used in the invention.

An MSC bank may be generated from the adipose tissue or bone marrow of healthy human subjects using known methods.

MSCs from various sources may be used. Suitably MSCs are from RoosterBio Inc. (5295 Westview Drive, Suite 275, Frederick, MD 21703, U.S.A.), cultured in RoosterBio Inc. media (RoosterNourish-MSC-CC, KT-021), and following the manufacturer's protocols for their culture. Other commercially available MSCs and/or media may be used, such as MSCs from NHSBT (NHS Blood and Transplant, 500 North Bristol Park, Filton, Bristol, BS34 7QH, U.K.), and/or and media from Miltenyi Biotec (MSC-Brew, 170-076-325, Miltenyi Biotec Ltd., Almac House, Church Lane, Bisley, Surrey GU24 9DR, UK) and/or Sartorius (MSC NutriStem XF Medium, 05-200-1A—Sartorius, Otto-Brenner-Straße 20, 37079 Goettingen, Germany).

We refer to Example 10 if any further guidance is required.

This priming step (sometimes called ‘education’ step) suitably comprises co-culturing the monocytes and MSCs.

The resulting primed monocyte cells are then analysed to confirm successful transformation of their phenotype. The cells may then be stored. These cells can then be used for administration to a subject over several weeks as a course of treatment, and/or as a one-off administration e.g. injection.

### Advantages

Prior art attempts to isolate functional cells have involved “super selecting” i.e, procedures designed to select cells occurring in the starting population at very low frequency. However, the number of cells obtained from such “super selection” methods is very low, which can be due to the fact of their extremely low frequency occurrence in the starting population. This is a problem in the art. It is an advantage of the invention that very high efficiency conversion from the starting cells to the functional triple positive cells of the invention is achieved. An advantage of this efficient conversion is that a very high proportion of the cells of the invention is produced from the starting population. A further advantage is that a very high absolute number of cells can be produced more easily than by using problematic “super selection” methods known in the art.

Thus, in one aspect the invention relates to a mixture of cells, said mixture comprising at least 50%, more suitably at least 80%, macrophages expressing MRC1, TIE2 and CD163.

### Method Steps

Suitably the starting cell comprises, or consists of, macrophage or monocyte. Most suitably the starting cell comprises, or consists of, monocyte.

When the starting cell is part of a population of cells, for example a population of mononuclear cells previously obtained from a sample of blood, suitably said population of cells comprises at least 20% macrophage or monocyte, most suitably monocyte.

Suitably said starting cell comprises, or consists of, PBMC.

Suitably said macrophage or monocyte is/are derived from PBMC.

Macrophage or monocyte may be isolated from PBMC by any suitable method known in the art such as immunomagnetic bead isolation or fluorescent-activated cell sorting, suitably using anti-CD14 or anti-CD45, more suitably anti-CD14.

A population of cells may be enriched for macrophage or monocyte by any suitable method known in the art such as immunomagnetic bead isolation or fluorescent-activated cell sorting, suitably using anti-CD14 or anti-CD45, more suitably anti-CD14.

### Culture Times

In the methods of the invention, the starting cells are monocytes, such as primary monocytes.

The invention does not embrace any method which starts with a macrophage (which may have technically been derived from blood, but in reality has already been in culture in vitro for a sustained period to differentiate it into a macrophage).

A primary monocyte is defined as a CD14-expressing monocyte that has just been isolated from the blood and not yet been placed in culture (i.e. it is still in suspension).

As soon as the monocyte is placed in a culture dish, it will stick (adhere) and is no longer a primary monocyte as it will start to slowly differentiate into a macrophage. In vitro incubated monocytes were primary monocytes prior to culture.

The methods of the invention expose primary monocytes directly to the MSCs at Day 0 i.e. culturing the primary monocytes with MSCs/contacting the primary monocytes with MSCs directly at Day 0.

Time in culture/Days in culture are counted from the time of placing the primary monocyte into culture.

Our cells are placed into culture immediately after isolation—the start time is the moment they are placed in culture. For example, if cells are isolated on a Monday AM and then placed in culture at 11 am, then Tuesday 11 am would be 24 hrs in culture (one day/Day 1).

Cells may be harvested and stored cryogenically (by freezing). Cells remain viable when thawed.

### Release Criteria

It will be noted that cells from different patients exhibit different levels of plasticity and/or different rates of change. Thus, the amount of time required in culture to induce the markers of interest may be longer for patients who are older or who exhibit other characteristics affecting the speed of change of expression in their mononuclear blood cells. Thus, in one embodiment suitably the method of the invention involves a “release” step. In this embodiment cells are only harvested (“released”) when specific predetermined criteria are met. For example, cells may be maintained in culture and a sample of those cells may be taken (or a sample being cultured separately in a smaller “buddy” culture in the same proportions may be taken) and tested and the cells may only be released to the next step of the method when they satisfy predetermined value(s) such as the proportion of cells expressing the triple markers MRC1, TIE2 and CD163. It may be noted that in the clinical trial data, sometimes fewer than three markers are assayed e.g. sometimes only MRC1 and CD163 are assayed. This is the ‘release assay’. This is an abbreviated assay which is used to ensure speed and reproducibility for the release criteria in the clinical setting (MHRA approved release assay). This release assay does NOT imply that the cells are ONLY positive for two of the three specified markers—in fact the cells we describe are positive for all three markers as claimed—it is merely an accepted practice in the field as endorsed/approved by the MHRA to produce an efficient and rapid ‘release assay’ which can be used to test + release clinical product—this is to be distinguished from the scientifically rigorous testing showing expression of all three markers as shown throughout this document. The release assay is a shortened version establishing that the product (cells of the invention) are in condition for clinical release—this is a different criterion to the scientific demonstration of expression of all three markers which remains true for the cells of the invention.

Notwithstanding this, suitably the invention requires culture for only seven days or less, more suitably culture for only five days or less.

It is an advantage of the invention that an autologous cell product is provided.

It will be noted that macrophage/monocytes do not actually expand in culture. Thus, in one aspect the method of the invention might comprise:
Collection of macrophage cells by leukapheresis or whole blood donation
Start culture with approximately two billion monocytes/macrophage cells (leukapheresis) or up to approximately 500 million monocytes/macrophage cells (whole blood donation)
Co-culture macrophage cells with mesenchymal stem cells
Assess phenotypic changes and harvest when at least 50%, more suitably at least 80%, of the macrophage cells express MRC1 and TIE2 and CD163.

### Methods of Medical Treatment

In one embodiment suitably the method comprises the step of separating macrophage from mesenchymal stem cells with which they were co-cultured.

Suitably macrophages are administered to a subject in need of same.

### Free Cells and Encapsulation

In one embodiment triple positive cells of the invention are injected into a patient. This may be referred to as “free cells”.

In one embodiment triple positive cells of the invention are encapsulated and injected into the patient encapsulated.

Suitably the composition of the invention is encapsulated. This has the advantage of improving retention and/or overcoming “wash-out” where cells are lost from the site of injection over time.

Suitably cells may be encapsulated using any suitable method known in the art.

It is an advantage of the invention that the cells of interest retain their triple positive characteristics when encapsulated. This is demonstrated in Example 4.

In one embodiment, cells are cultured for 3 to 7 days, more suitably for 3 days, and then introduced back into the patient.

In one embodiment, cells are encapsulated immediately, the encapsulated cells are cultured, for example for 3 to 7 days, more suitably for 3 days, and the encapsulated cells are then reintroduced to the patient.

In this embodiment, there is optionally no separation of the triple positive cells from the MSCs with which they are co-cultured in order to induce triple positive expression.

Thus, in one embodiment, the monocytes/macrophages and the MSCs may be co-encapsulated. More suitably the monocytes/macrophages and the MSCs are co-encapsulated in a 3:1 ratio. To the best of the inventors' knowledge, this is the first disclosure of encapsulation of monocytes/macrophages and MSCs together in a single encapsulation.

In one embodiment suitably the monocytes/macrophages are encapsulated. In one embodiment the invention provides an encapsulation, within which are contained triple positive monocyte/macrophage cells. In one embodiment said encapsulation further comprises MSCs. When the encapsulation comprises both triple positive monocyte/macrophage cells and MSCs, suitably they are present in a 3:1 ratio of monocyte/macrophage cells:MSCs. Most suitably they are present in a 3:1 ratio of triple positive monocyte/macrophage cells:MSCs.

### Negative Markers

Suitably cells produced according to the method of the invention have downregulated expression (i.e. lower expression compared to primary monocytes cultured alone and/or compared to primary monocytes uncultured) of the following markers:
MMP-9 (accession: P14780)
NRP1 (accession: 014786—also known as CD304)
HB-EGF (accession: P01133)

With regard to MMP-9, this marker is downregulated following co-culture with MSCs, whereas it is also upregulated if monocytes are cultured without MSCs.

In one embodiment a cell is considered not to express a particular marker when said marker is undetectable.

Detection/assessment of downregulation is suitably assessed using mean fluorescent intensity i.e. the amount of protein expressed. Suitably this is expressed as a percentage of the level measured on primary monocytes i.e. monocytes isolated from peripheral blood which have not been cultured in vitro/ex vivo. A protein is considered downregulated when expressed (detected) at a level lower than the level on primary monocytes.

Suitably a protein is considered downregulated when expressed (detected) at a statistically significant level lower than the level on primary monocytes. For MMP-9 suitably there is a median 60% reduction in expression when the monocytes are primed with MSCs. We have carried out further statistical analysis to show that we that 50% of cells will downregulate MMP-9 by at least this 60% value when co-cultured with MSCs. For HB-EGF, suitably there is a 30% reduction in the expression if this protein when we co-culture monocytes with MSCs. Suitably 15% of cells will be at least 30% downregulated for HB-EGF every time.

### Sequence Identity

When assessing expression of markers as described herein, detection of the marker may be found when the precise sequence is identified, or when it is considered that the gene/protein of interest is indeed expressed. For example, allelic variants or normal genetic variability between individuals in a species is a well known phenomenon and (for example) minor or negligible sequence differences between a marker in a subject and the reference sequence being assessed will not affect the assessment of whether the marker is expressed or not expressed in a sample/in a cell.

It may be desired to consider sequence relationships in terms of sequence identity. Sequence comparisons can be conducted by eye or, more usually, with the aid of readily available sequence comparison programs. These publicly and commercially available computer programs can calculate percent homology (such as percent identity) between two or more sequences.

Percent identity may be calculated over contiguous sequences, i.e., one sequence is aligned with the other sequence and each amino acid in one sequence is directly compared with the corresponding amino acid in the other sequence, one residue at a time. This is called an “ungapped” alignment. Typically, such ungapped alignments are performed only over a relatively short number of residues (for example less than 50 contiguous amino acids).

Although this is a very simple and consistent method, it fails to take into consideration that, for example in an otherwise identical pair of sequences, one insertion or deletion will cause the following amino acid residues to be put out of alignment, thus potentially resulting in a large reduction in percent homology (percent identity) when a global alignment (an alignment across the whole sequence) is performed. Consequently, most sequence comparison methods are designed to produce optimal alignments that take into consideration possible insertions and deletions without penalising unduly the overall homology (identity) score. This is achieved by inserting “gaps” in the sequence alignment to try to maximise local homology/identity.

These more complex methods assign “gap penalties” to each gap that occurs in the alignment so that, for the same number of identical amino acids, a sequence alignment with as few gaps as possible—reflecting higher relatedness between the two compared sequences—will achieve a higher score than one with many gaps. “Affine gap costs” are typically used that charge a relatively high cost for the existence of a gap and a smaller penalty for each subsequent residue in the gap. This is the most commonly used gap scoring system. High gap penalties will of course produce optimised alignments with fewer gaps. Most alignment programs allow the gap penalties to be modified. However, it is preferred to use the default values when using such software for sequence comparisons. For example when using the GCG Wisconsin Bestfit package (see below) the default gap penalty for amino acid sequences is −12 for a gap and −4 for each extension.

Calculation of maximum percent homology therefore firstly requires the production of an optimal alignment, taking into consideration gap penalties. A suitable computer program for carrying out such an alignment is the GCG Wisconsin Bestfit package (University of Wisconsin, U.S.A; Devereux et al., 1984, Nucleic Acids Research 12:387). Examples of other software than can perform sequence comparisons include, but are not limited to, the BLAST package, FASTA (Altschul et al., 1990, J. Mol. Biol. 215:403-410) and the GENEWORKS suite of comparison tools.

Although the final percent homology can be measured in terms of identity, the alignment process itself is typically not based on an all-or-nothing pair comparison. Instead, a scaled similarity score matrix is generally used that assigns scores to each pairwise comparison based on chemical similarity or evolutionary distance. An example of such a matrix commonly used is the BLOSUM62 matrix—the default matrix for the BLAST suite of programs. GCG Wisconsin programs generally use either the public default values or a custom symbol comparison table if supplied. It is preferred to use the public default values for the GCG package, or in the case of other software, the default matrix, such as BLOSUM62. Once the software has produced an optimal alignment, it is possible to calculate percent homology, preferably percent sequence identity. The software typically does this as part of the sequence comparison and generates a numerical result.

### Reference Sequence/Database Release

Sequences deposited in databases can change over time. Suitably the current version of sequence database(s) are relied upon. Alternatively, the release in force at the date of filing is relied upon.

As the skilled person knows, the accession numbers may be version/dated accession numbers. The citeable accession numbers for the current database entry are the same as herein, but omitting the decimal point and any subsequent digits.

GenBank is the NIH genetic sequence database, an annotated collection of all publicly available DNA sequences (National Center for Biotechnology Information, U.S. National Library of Medicine 8600 Rockville Pike, Bethesda MD, 20894 USA; Nucleic Acids Research, 2013 January; 41 (D1): D36-42) and accession numbers provided relate to this unless otherwise apparent. Suitably the current release is relied upon. More suitably the release available at the effective filing date is relied upon. Most suitably the GenBank database release referred to is NCBI-GenBank Release 241:15 Dec. 2020.

Suitably the current version of sequence database(s) are relied upon. Alternatively, the release in force at the date of filing is relied upon. For the avoidance of doubt, the UniProt consortium European Bioinformatics Institute (EBI), SIB Swiss Institute of Bioinformatics and Protein Information Resource (PIR)'s UniProt Knowledgebase (UniProtKB) Release 2021_01 published 10 Feb. 2021 is relied upon. UniProt (Universal Protein Resource) is a comprehensive catalogue of information on proteins (“UniProt: the universal protein knowledgebase” Nucleic Acids Res. 45: D158-D169 (2017)).

### Further Applications

The properties of the primed Mo/MΦs of the invention may be affected by cryopreservation. The inventors have data showing that the viability of Mo/MΦs of the invention is not affected following cryopreservation and storage. Whether the efficacy of these cells in revascularising ischaemic tissue is altered following storage may be monitored by the skilled person and the storage and/or dose of cells administered to a subject may be altered accordingly if necessary.

Suitably cells/cell populations of the invention are produced within a GMP facility.

The invention may be used in treatment of peripheral vascular disease and/or peripheral arterial disease (suitably patients with intermittent claudication and/or chronic limb threatening ischaemia). In this situation, the cells stimulate the sprouting of new vessels (angiogenesis) as well as induce positive remodelling of existing vessels (arteriogenesis).

The invention may be used as a tissue remodelling, anti-fibrotic therapy for example to treat conditions that are associated with tissue fibrosis, including fibrotic lung, kidney, liver and skin disease.

The invention may be used to treat ischaemic heart disease or heart failure as a consequence of fibrosis. For ischaemic heart disease, the invention may be used to stimulate angiogenesis and/or arteriogenesis. In heart failure that occurs as a consequence of fibrosis, the invention may be used to induce positive tissue remodelling, reduce fibrosis and/or improve heart muscle function.

The invention may be used to treat ischaemic stroke, where the invention induces new blood vessel growth by way of angio- or arteriogenesis.

The invention may be used to treat ischaemic tissue such as ischaemic heart tissue or ischaemic brain tissue.

### Demonstration of Effects

The invention may be used in treatment of peripheral vascular disease, suitably peripheral arterial disease, suitably CLI or chronic limb threatening ischaemia (CLTI). Data in this application supports this use e.g. FIG. 15A-D shows significantly increased smooth muscle cell proliferation when the cells are exposed to the conditioned medium generated from the invention, FIG. 16A,B shows significantly improved revascularisation following delivery of the invention with evidence of in vivo arteriogenesis (FIG. 16C,D). Thus the reader will appreciate from the demonstration of smooth muscle proliferation in vitro, improved limb perfusion following delivery of the invention into the ischaemic limbs of mice and a resulting larger number of vessels seen in vivo that that the invention will address ischaemia because these features result in increased blood supply to the ischaemic tissue.

The invention may be used in treatment of fibrosis. Data in this application supports this use e.g. FIG. 19 shows that the invention produces higher levels of anti-fibrotic proteins HGF, IL-10 and TNFα compared with monocytes that are not primed. FIG. 20 shows that the invention reverses fibrosis in vitro by way of reduced SMA and fibronectin expression on stimulated fibroblasts. FIG. 21A shows the invention protects small airway epithelial cells from undergoing apoptosis and FIG. 21B shows that the invention improves endothelial proliferation. FIG. 22 shows a reduction in fibrosis in vitro (collagen staining) when the invention is delivered into tissue after injury. Thus the reader will appreciate from the demonstration of reduced fibrosis in vitro and in vivo as well as improved small airway epithelial cell and endothelial cell survival (epithelial and endothelial injury and death occur during the fibrotic process) that the invention will address fibrosis by reducing fibroblast to mesenchymal transition and improving epithelial and endothelial cell survival.

The invention may be used in induction of revascularisation. Data in this application supports this use e.g. FIG. 16A shows improved limb perfusion in mice that are treated with the invention following induction of hindlimb ischaemia; also FIGS. 16C and D shows that the muscle contains larger arterioles, suggesting vessel remodelling and arteriogenesis. The invention also produces higher levels of HGF than cells cultured alone (FIG. 19) and this protein is known to stimulate the revascularisation of ischaemic tissues Thus the reader will appreciate from the demonstration of improved limb perfusion and arteriogenesis that the invention will induce revascularisation because angiogenesis and/or arteriogenesis are shown, and these processes directly result in revascularisation of ischaemic tissue by way of inducing better collateralisation.

The invention may be used in treatment of ischaemic stroke. Data in this application supports this use e.g. FIGS. 15 and 16 which show increased in vitro smooth muscle cell proliferation and in vivo arteriogenesis respectively. In addition, FIG. 21B shows increased vascular endothelial cell proliferation (angiogenesis). Thus the reader will appreciate from the demonstration of increased angio- and arteriogenesis that the invention will address ischaemic stroke because new blood vessel formation in the ischaemic brain will allow reperfusion of previously ischaemic tissue from the stroke.

### Further Embodiments

In one embodiment the invention provides a method of treating a mammalian subject comprising administering a cell as described above to said subject.

In one embodiment the invention provides a method of treating a mammalian subject comprising administering a population of cells as described above to said subject.

In one embodiment the invention provides mesenchymal stem cell-primed monocytes for tissue remodelling.

Further particular and preferred aspects are set out in the accompanying independent and dependent claims. Features of the dependent claims may be combined with features of the independent claims as appropriate, and in combinations other than those explicitly set out in the claims.

Where an apparatus feature is described as being operable to provide a function, it will be appreciated that this includes an apparatus feature which provides that function or which is adapted or configured to provide that function.

The invention is now described by way of numbered paragraphs:

Paragraph 1. A population of cells, wherein said population of cells comprises at least 50% macrophage or monocyte cells, characterised in that at least 50% of said macrophage or monocyte cells express each of the markers: MRC1; TIE2; and CD163.

Paragraph 2. A population of cells according to Paragraph 1, wherein said population of cells comprises at least 70% macrophage or monocyte cells.

Paragraph 3. A population of cells according to Paragraph 1 or Paragraph 2, wherein said population of cells comprises at least 80% macrophage or monocyte cells.

Paragraph 4. A population of cells according to any of Paragraphs 1 to 3 wherein at least 60% of said macrophage or monocyte cells express each of the markers: MRC1; TIE2; and CD163.

Paragraph 5. A population of cells according to any preceding Paragraph wherein said macrophage or monocyte cells express human 25f9.

Paragraph 6. A population of cells according to any preceding Paragraph wherein said macrophage or monocyte cells express CD14 and/or CD45.

Paragraph 7. A population of cells according to any of Paragraphs 1 to 6 comprising a 3:1 ratio of monocyte/macrophage cells:MSCs.

Paragraph 8. A population of cells according to Paragraph 7 comprising a 3:1 ratio of triple positive monocyte/macrophage cells:MSCs.

Paragraph 9. A population of cells according to any of Paragraph 1 to 8 wherein said cells are encapsulated.

Paragraph 10. A method comprising (a) providing a macrophage or monocyte from a subject; (b) providing a MSC; (c) culturing said macrophage or monocyte with said MSC.

Paragraph 11. A method according to Paragraph 10 wherein step (c) comprises contacting said macrophage or monocyte with said MSC to produce a cell mixture, and culturing said cell mixture.

Paragraph 12. A method according to Paragraph 10 or Paragraph 11 wherein the ratio of (macrophages or monocytes):(MSCs) in step (c) is 3:1.

Paragraph 13. A method according to any of Paragraphs 10 to 12 wherein the cells are cultured for about 3 to 7 days, preferably for about 3 to 5 days.

Paragraph 14. A method according to Paragraph 13 wherein the cells are cultured for about 3 days.

Paragraph 15. A method according to any of Paragraphs 10 to 13 wherein the cells are cultured in a medium, and wherein said medium is changed every 5 days.

Paragraph 16. A population of cells according to any of Paragraphs 1 to 9 for use in treatment of peripheral vascular disease, suitably chronic limb threatening ischaemia.

Paragraph 17. A population of cells according to any of Paragraphs 1 to 9 for use in treatment of fibrosis.

Paragraph 18. A population of cells according to any of Paragraphs 1 to 9 for use in treatment of ischaemic stroke.

Paragraph 19. A method of treating a mammalian subject comprising administering a population of cells according to any of Paragraphs 1 to 9 to said subject.

Paragraph 20. A method according to Paragraph 19 comprising administering a dose of about 10⁶-10⁹ said cells.

Paragraph 21. Use of a population of cells according to any of Paragraphs 1 to 9 to induce angiogenesis in a mammal.

### BRIEF DESCRIPTION OF THE DRAWINGS

The present invention is now described further, with reference to the accompanying drawings, in which:

FIG. 1 shows comparative data: no increase on TIE2 or CD163 expression using the methods of Rybalko et al 2017, despite an increase in the expression of CD206.

FIG. 2 shows a diagram of marker expression in MSC-primed monocytes (cells/cell population according to the present invention—see (B)) compared to control cells (not the invention—see (A)).

FIG. 3 shows a bar chart.

FIG. 4 shows bar charts.

FIG. 5 shows plots

FIG. 6 shows plots and graphs

FIG. 7 shows plots and graphs

FIG. 8 shows tables (heat maps)

FIG. 9 shows bar charts

FIG. 10 shows plots

FIG. 11 shows Venn diagrams. FIG. 11B numbers are percentage expression of Mo/MΦs—69.96% triple positive—n=21.

FIG. 12 shows graphs

FIG. 13 shows bar charts

FIG. 14 shows Venn diagrams

FIG. 15 shows graphs and bar charts

FIG. 16 shows a graph, photographs and plots

FIG. 17 shows plots

FIG. 18 shows graphs

FIG. 19 shows graphs

FIG. 20 shows graphs

FIG. 21 shows a plot and a graph

FIG. 22 shows photographs and a graph

FIG. 23 shows a graph. The straight line at the top (‘100’) is the data—100% of the mice survived at all time points.

FIG. 24 shows a bar chart.

FIG. 25 shows bar charts.

FIG. 26 shows plots.

FIG. 27 shows plots. Cells of the invention. n=7/group (A) Day 3: No difference in IL-12 expression (IL-12 is still highly expressed on our cells after co-culture) (B) Day 7: No difference in IL-12 expression (IL-12 is still highly expressed on our cells after co-culture).

FIG. 28 shows a bar chart. TIE2 expression is highest at day 3 of co-culture. Significant increase in TIE2 expression at day 3, which starts to fall by Day 7. After this, TIE2 continues to fall to levels similar to control cells (P<0.0001 using Kruskal-Wallis test with post-hoc Dunn's multiple comparison text, n=6 samples, error bars are SEM)

FIG. 29 shows CD206 expression is highest at day 3 of co-culture. Significant increase in CD206 expression at day 3, which starts to fall by Day 7. After this, CD206 continues to fall to levels similar to control cells (P<0.0001 using Kruskal-Wallis test with post-hoc Dunn's multiple comparison text, n=6 samples, error bars are SEM)

FIG. 30 shows CD163 expression is highest at day 3 of co-culture. Significant increase in CD163 expression at day 3, which starts to fall by Day 7. After this, CD163 continues to fall to levels similar to control cells (P<0.0001 using Kruskal-Wallis test with post-hoc Dunn's multiple comparison text, n=6 samples, error bars are SEM)

FIG. 31 shows graphs (i, ii, v, vi) and photographs (iii, iv). These show results from a human study demonstrating effectiveness of the invention in human subjects. We refer to Example 15 for more details.

### EXAMPLES

Although illustrative embodiments of the invention have been disclosed in detail herein, with reference to the accompanying drawings, the invention is not limited to those precise embodiments. Various changes and modifications can be effected therein by one skilled in the art without departing from the scope of the invention as defined by the appended claims and their equivalents.

### Example 1—Comparative Data

Rybalko et a/2017 (Regen. Med. Volume 12, number 2, pages 153-167) showed an increase in CD206 expression on U-937 cells following co-culture with bone marrow derived MSCs. We compared our method of priming monocytes with theirs by following their methodology.

U-937 cells (CRL-1593.2, ATCC) were cultured in in RPMI-1650 growth media supplemented with 10% FCS and 1% penicillin-streptomycin. For the co-culture experiments, MSCs were seeded in 6-well transwell inserts at 20,000 cells/cm² in DMEM containing 10% FCS and cultured under standard cell culture conditions over night to allow for adherence. U-937 cells were treated with 100 nM of 12-O-tetradecanoylphorbol-13-acetate (TPA) for 48 hours and seeded in 6-well plates at a concentration of 400,000 cells/well in RPMI media containing 10% FCS. Following overnight culture, the media were replaced with serum-free DMEM or RPMI media/10% FCS for the stem cells and U-937 cells respectively. The transwell inserts containing the MSCs were transferred to the 6-well plates containing U-937 cells. For direct co-culture experiments, U-937 cells were treated with 100 nM of TPA for 48 hours and seeded in 6-well plates at a concentration of 400,000 cells/well in RPMI/10% FBS. The MSCs were then added at 1×10⁵ cells/well. Transwell and directly cultured U-937 cells were analysed by flow cytometry.

The above method confirmed that we had reproduced the data/methodology disclosed in Rybalko et al by achieving an increase in CD206 expression to 67.6% and 69.2% following direct and transwell co-culture respectively of U-937 cells on MSCs.

However, this method resulted in only 3.1% and 0.7% TIE2-expression and 18.1% and 5.5% CD163-expression following direct and transwell co-culture respectively (FIG. 1).

This amounted to a “triple-positive” expression of only 1% and 2% for directly and transwell co-cultured cells respectively.

Thus these comparative data show that the method disclosed in Rybalko et al., does not generate the cells/cell populations of the invention.

### Example 2—Antifibrotic Experiments

In one embodiment the invention provides use of a cell composition as described herein in treatment of fibrosis. Data presented herein show excellent effects in the leg model. The skilled person would expect that this makes it plausible and credible that the treatment works in fibrosis of the lung. Indeed, based on the evidence provided in this document, the MHRA (Medicines and Healthcare Products Regulatory Agency of the UK Government) has moved forward with the clinical application in the lung. This illustrates that the exemplary data provided herein support the use of the cell compositions described in a range of antifibrotic clinical applications.

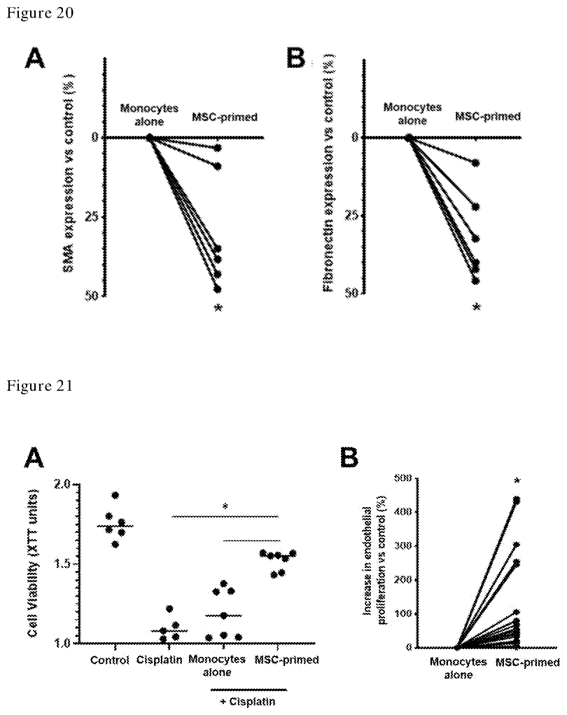

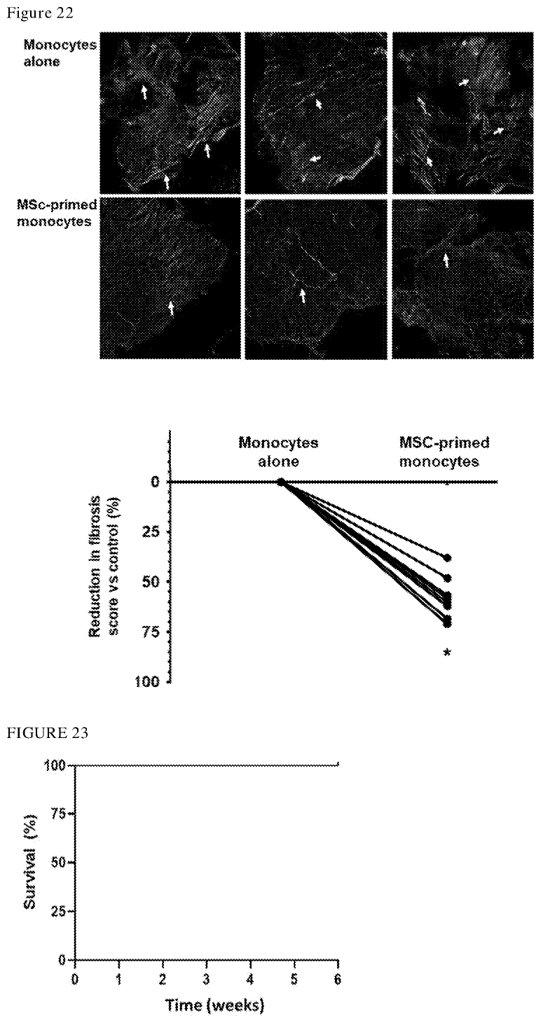

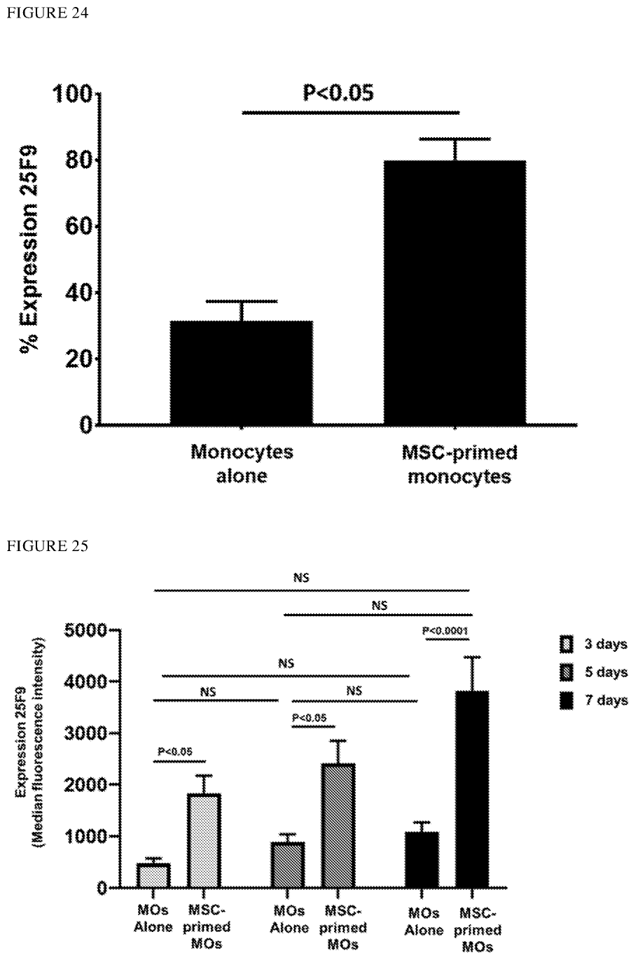

MSC-primed monocytes have anti-fibrotic activity in vitro. These cells (conditioned media) stimulate significant abrogation of SMA (FIG. 20A) and fibronectin (FIG. 20B) expression (markers of fibrosis) following stimulation of human lung fibroblasts with TGFβ). The cells of the invention also rescue small airway epithelial cells (SAECs) from cisplatin induced apoptosis (FIG. 21A) and stimulate 3-fold greater endothelial survival compared with whole population monocyte (FIG. 21B). Regeneration of normal endothelium and vascular remodelling is critical to healthy repair and resolution of fibrosis following lung injury.

MSC primed monocytes (i.e. cells of the invention) have potent anti-fibrotic function in vivo: Delivery of these cells into the murine hindlimb following induction of ischaemia results in significantly reduced tissue fibrosis compared with delivery of monocytes that have been cultured alone.

### Example 3—Clinical Safety Study

Five patients are injected with cells according to the invention.

Patients are followed and assessments of:
Lung function
CT Scans
6 minute walk test
QoL questionnaires
   are made.

### Example 4—Encapsulation

In one embodiment the population of cells of the invention is encapsulated. In this example we show encapsulation of monocytes with MSCs

Human monocytes and MSCs were prepared as 3:1 mixture and encapsulated using an alginate solution (1.5% w/v prepared in 0.9% w/v NaCl) and CaCl₂ as per our previously published technique (Ludwinski FE, Patel AS, Damodaran G, et al. Encapsulation of macrophages enhances their retention and angiogenic potential. NPJ Regen Med. 2019; 4:6). Capsules were washed twice with Hank's Balanced Salt Solution (HBSS) through a 70 μM cell strainer (Corning, UK) and transferred to 6-well plates containing 2 ml of RPMI/10% FCS media for 3 days. After this time, cells were extracted into single cell suspensions using trypsin/EDTA and analysed via flow cytometry for the expression of TIE2, CD163 and CD206.

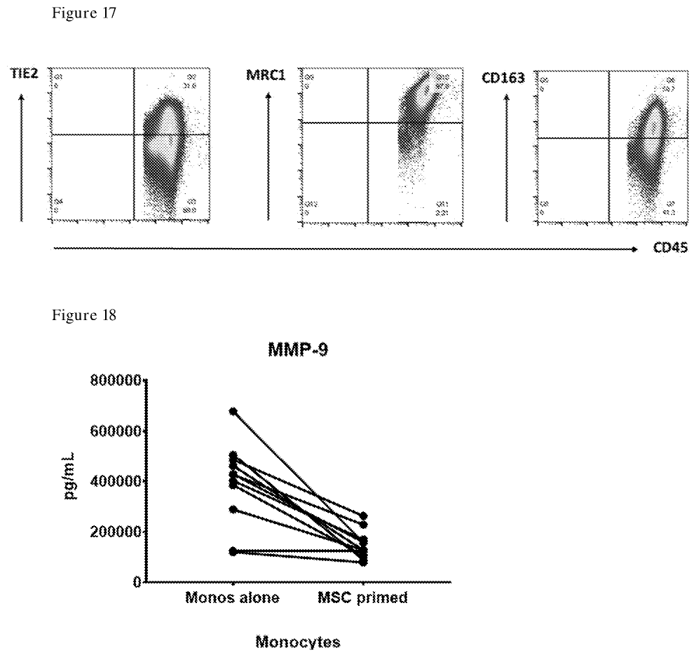

Encapsulation of the monocytes with MSCs results in an increase in TIE2 (31%), CD206 (97.8%) and CD163 (58.7%), suggesting a novel method for co-encapsulating cells for delivery rather than the need for co-culture (FIG. 17).

FIG. 17 shows alginate encapsulation of primary monocytes with MSCs. Monocytes exhibit high expression of TIE2, CD206 and CD163 following encapsulation without the need for adherent co-culture.

It is an advantage of the invention that the population of cells retain their triple positive characteristics when encapsulated. This is demonstrated in FIG. 17.

### Example 5—Production

Monocytes are isolated from the blood of patients with CLI.

In their steady state these monocytes have no ability to salvage the ischaemic limbs of mice in our pre-clinical models.

These monocytes are co-cultured with MSCs.

MSCs may be obtained from any suitable source. In this example, MSCs are used from an in-house bank of adipose-derived MSCs.

The co-culturing is carried out to drive/prime these monocytes to upregulate their expression of 3 markers: TIE2, MRC1 and CD163. The presence of the combination of these markers gives these cells the ability to then stimulate the growth of new blood vessels in order to salvage the ischaemic limbs of mice.

Important steps disclosed herein have been to optimise co-culture conditions by determining
the optimal ratio of monocyte:MSC
and
the length of time needed in culture to stimulate a ˜20-fold increase (eventually comprising >80% of the whole monocyte population) in the number of Mo/MΦs that express all three of these markers.

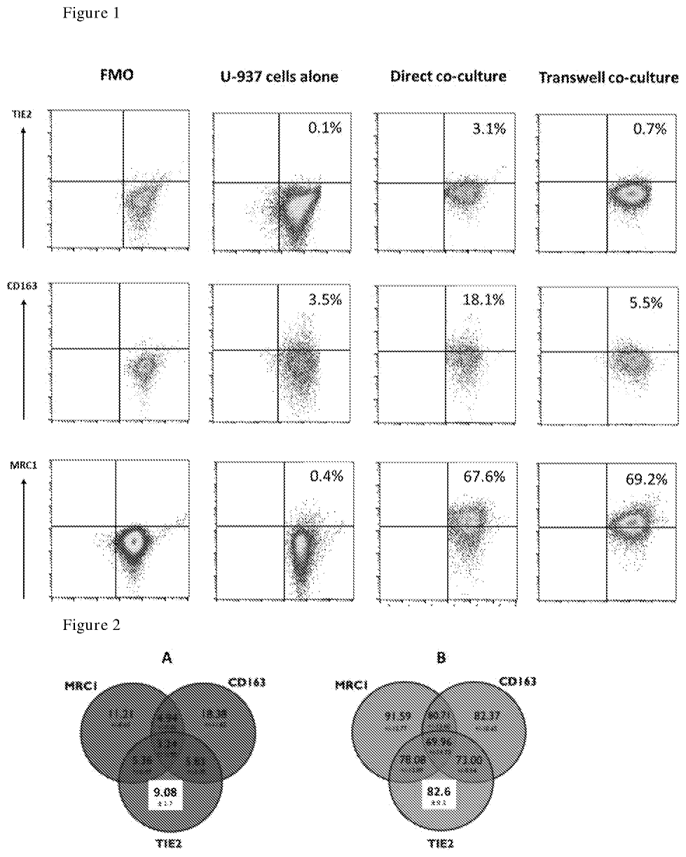

We refer to FIG. 2 which shows Over 20-fold increase in triple-positive expressing monocytes following co-culture with MSCs under our optimal conditions (B) compared with culture of monocytes without MSCs (A).

### Example 6—Optimised Production

We have optimised the conditions required to engineer triple expressing TIE2+, MRC1+, CD163+Mo/MΦs from whole population monocytes isolated from either patients with CLI or from controls.

We have tested the functional capacity of these engineered cells to show they have the ability to regulate the growth of blood vessels.

Functional in vitro assays show that the conditioned medium from these MSC-primed Mo/MΦs increases arteriogenesis (smooth muscle cell proliferation) compared with conditioned medium from monocytes cultured without MSCs (FIG. 3). Testing the conditioned medium is to show that the proteins the MSC-primed monocytes are producing are functional.

FIG. 3 shows that MSC-primed Mo/Mos stimulate greater proliferation of smooth muscle cells compared with MOs alone. *P<0.05, n=8; SMC=smooth muscle cell.

### Example 7—Revascularisation Using Cells of the Invention

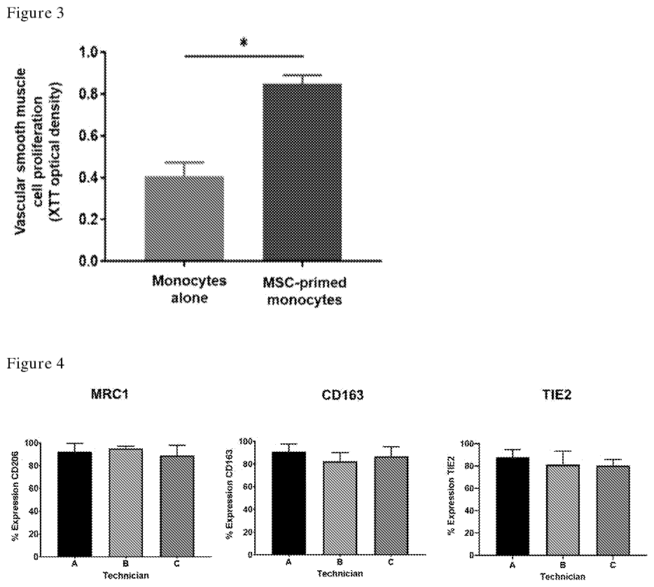

The MSC-primed Mo/MΦs of the invention have a significantly greater capacity to revascularise the ischaemic limb in our murine model of hindlimb ischaemia HLI compared with non-primed control Mo/MΦs (FIG. 4).

FIG. 16A shows significantly greater revascularisation of the ischaemic hindlimb with MSC-primed MOs compared with MOs alone (P<0.001, *P<0.001. **P<0.05 by post-hoc tests, n=8 mice/group).

### Example 8

### Methods

### Patient Recruitment

Patients with CLI (Rutherford classification 4-6) who presented with lower limb tissue loss and/or rest pain, controls (age-matched with no clinical evidence of peripheral vascular disease) and healthy controls were recruited. Venous blood was collected in ethylenediaminetetraacetic acid (EDTA) tubes (BD Vacutainer, UK). The isolation of monocytes from whole blood is a well-established technique in our department. In short, firstly PBMCs were isolated from 100 mLs of venous blood using Ficoll-Paque and magnetic immunobeads. Blood was first was mixed 1:1 with RPMI-1640 before layering on Ficoll-Paque reagent in the ratio of 2:1 (Blood: Ficoll-Paque). Samples were centrifuged at 400 g for 30 mins and the mononuclear removed. Remaining erythrocytes were lysed using BD Pharm Lyse (BD Biosciences). Following a wash step, the resultant cell suspension was blocked using FcR blocking reagent (Miltenyi Biotec) and incubated with anti-human CD14 microbeads followed by immunomagnetic positive selection. Isolated monocytes were labelled with FITC-conjugated anti-human CD14 (BD Biosciences) and viability staining dye (7-AAD; BD Biosciences) prior to analysis using flow cytometry (Attune, Thermo UK) to determine the purity and viability of the isolated cells.

### Primary Cell Culture

Primary human adipose-derived mesenchymal stem cells (adMSCs) which have already been isolated, pooled and banked by our team within the Academic Department of Vascular Surgery, King's College London, have been used in this study. These adMSCs were maintained in Dulbecco's Modified Eagle Medium (DMEM) supplemented with 10% foetal calf serum (FCS, ThermoFisher Scientific), 1% Antibiotic/Mycotic (ThermoFisher Scientific), 5 ng/mL Epidermal Growth Factor (EGF) (R&D Systems), 1 ng/ml basic Fibroblast Growth Factor (bFGF), and 0.25 ng/mL Transforming Growth Factor (TGFβ, Millipore).

Isolated monocytes were cultured in monocyte media (RPMI-1640 supplemented with 10% FCS and 1% anti-biotic/-mycotic). All cells were cultured in a humidified incubator at 37° C., 5% CO₂.

Co-Culture Technique of Primary Monocytes with adM SCs

For co-culture experiments, frozen adMSCs were first thawed and seeded 2-3 days in their regular culture conditions until they were 80-90% confluent. Once confluency was achieved, adMSC media was removed and primary isolated monocytes seeded at a range of co-culture ratios in monocyte media. 10-15% more monocytes were added than the final number required as experience from our laboratory indicates that ˜10% of cells will not stick (see table below). Media was replaced at day 1 and then every 48 hours up until harvesting.

TABLE 1
Seeding densities required to achieve
target monocytes:adMSCs ratios

| | Number of cells per | | Volume of |
|---|---|---|---|
| Ratio of | flask | | RPMI-1640 |
| monocytes:adMSCs | (monocytes:adMSCs) | Size of flask | media |
| 1:1 | 400 000:300 000 | T25 | 5 mL |
| 2:1 | 700,000:300 000 | T25 | 5 mL |
| 3:1 | 1.2 × 10⁶:300 000 | T25 | 5 mL |
| 5:1 | 3 × 10⁶:500 000 | T75 | 10 mL |

### Collection of Conditioned Media

Monocytes in co-culture were magnetically re-separated from the adMSCs using anti-CD45 beads (Miltenyi Biotec). These monocytes that had been primed with adMSCs and those cultured alone were re-seeded into T25 flasks in RPMI containing 10% FCS. Conditioned media was collected from these flasks containing after 48 hours of culture. The media was and centrifuged at 400 g for 5 minutes to remove dead/floating cells. The supernatant was re-centrifuged at 25000 g for tomins. The supernatant was snap frozen and stored at −80° C.

Flow Cytometric Analysis of Cells e.g. Monocytes

### Extracellular Phenotyping

For flow cytometric analysis, cells were detached using Trypsin-EDTA (Sigma Aldrich). Cell suspensions were centrifuged at 400 g for 5 minutes before washing in wash buffer (PBS, 2 mM EDTA, 0.5% BSA). Cell suspensions were blocked using human FeR blocking reagent for 20 minutes, after which the panel of fluorescently conjugated antibodies (Table 3) were added and cells incubated in the dark at 4° C. for 30 mins before washing. Cells were analysed on an Attune™ NxT (ThermoFisher) flow cytometer using Attune™ NxT software v2.7.0. Compensation was performed with OneComp eBeads (ThermoFisher). For every sample, fluorescence minus-one (FMO) controls were used to determine negative staining. The gating strategy involved removal of debris, doublets and dead cells followed by gating on CD45^{+ve} cells to identify the monocytes.

TABLE 2
List of antibodies used for extracellular
staining of cells e.g. monocytes.

| Antibody/ | | | Marker/ | Accession |
|---|---|---|---|---|
| Probe | Fluorochrome | Supplier | Protein | Number |
| Anti-human | BV421 | BD | human CD45 | See above |
| CD45 | | Biosciences | | |
| Anti-human | BB515 | BD | human CD206 | See above |
| CD206 | | Biosciences | (MRC1) | |
| Anti-human | BV711 | BD | human CD163 | See above |
| CD163 | | Biosciences | | |
| Anti-human | BV605 | BD | human CD105 | Uniprot |
| CD105 | | Biosciences | | P17813 |
| Anti-human | APC-H7 | BD | human CD80 | Uniprot |
| CD80 | | Biosciences | | P33681 |
| Anti-human | PE-Cy7 | Invitrogen | human CD38 | Uniprot |
| CD38 | | | | P28907 |
| Anti-human | PE | R&D | human TIE2 | See above |
| TIE2 | | | | |
| Anti-human | APC | R&D | human | Uniprot |
| VEGFR1 | | | VEGFR1 | P17948 |
| Live-Dead | 7-AAD | BD | Live-Dead | |
| discriminator | | Biosciences | discriminator | |

### Intracellular Phenotyping

Monocytes were centrifuged at 400 g for 5 minutes, supernatant discarded and cells resuspended in 4% paraformaldehyde (PFA) for 20 minutes for fixation. Fixed samples were washed in wash buffer, centrifuged at 400 g for 5 minutes and the supernatant discarded. Cell pellets were resuspended in 100 μL wash buffer and incubated with anti-human CD45 in the dark at 4° C. for 30 minutes. Samples were washed in wash buffer before being centrifuged and resuspended in 100 μL perm/wash Buffer (BD Biosciences) for cell permeabilisation. Samples were stained with the intracellular panel for 30 minutes (Table 3). Samples were washed in perm/wash buffer before being resuspended for analysis.

TABLE 3
List of antibodies used for intracellular
staining of cells e.g. monocytes.

| Antibody/ | | | Marker/ | Accession |
|---|---|---|---|---|
| Probe | Fluorochrome | Supplier | Protein | Number |
| Anti-human | PE-Cy7 | BD | human CD45 | See above |
| CD45 | | Biosciences | | |
| Anti-human | AlexaFluor 405 | R&D | human | Uniprot |
| VEGF | | | VEGF | P15692 |
| Anti-human | APC | R&D | human HB- | Uniprot |
| HB-EGF | | | EGF | Q99075 |
| Anti-human | Vi0515 | Miltenyi | human IL-10 | Uniprot |
| IL-10 | | Biotec | | Q13651 |
| Anti-human | PE | eBioscience | human IL-12 | Uniprot |
| IL-12 | | | | P42701 |
| Anti-human | AlexaFluor 700 | eBioscience | human TNF-α | Uniprot |
| TNF-α | | | | P01375 |

### Smooth Muscle Cell Proliferation Assays

Human smooth muscle cells (SMCs, Lonza, UK) were plated at 1×10³ cells/well in a 96-well plate in RPMI-1640 medium containing 10% FCS for 24 hrs. Cells were then incubated with conditioned medium (CM) overnight for 24 hours. Control wells consisted of unconditioned RPMI-1640 media alongside wells containing media alone for background subtraction. After 24 hours, XTT (1 mg/ml) was added to each well and the plate incubated for 4 hours. Following this, absorbance was measured at 450 nm using a plate reader (Filter Max F5, Molecular Devices) with a reference wavelength of 620 nm. Readings were taken every 2 hours until 12 hours then at 22 hours and two-hourly for another 14 hours (36 hours in total). The assay was performed in duplicate for 11 individual samples of day 3 and 7 primed/non-primed monocytes. All data were blinded and independently analysed.

### Statistical Analysis

Data were analysed using GraphPad Prism version 8 (GraphPad Inc.), Non-parametric tests were used for all experiments (Mann-Whitey U for unpaired and Wilcoxon matched-pairs signed rank test for paired data). Data are presented as mean±standard error of the mean (SEM) unless otherwise stated.

### Results

### Phenotype of adM SC-Primed Monocytes

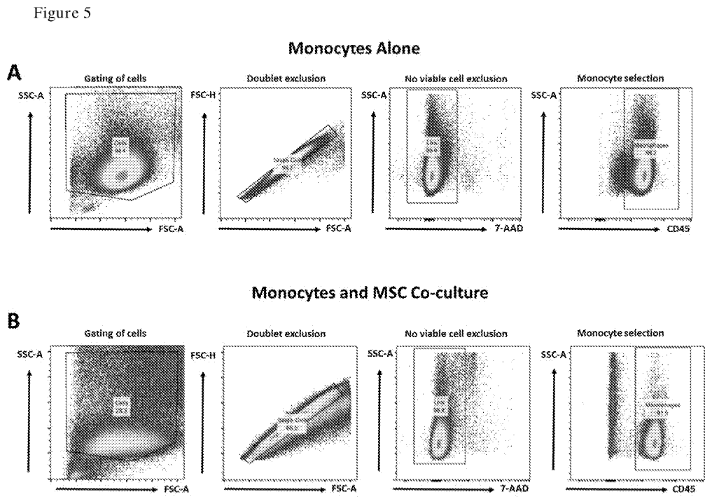

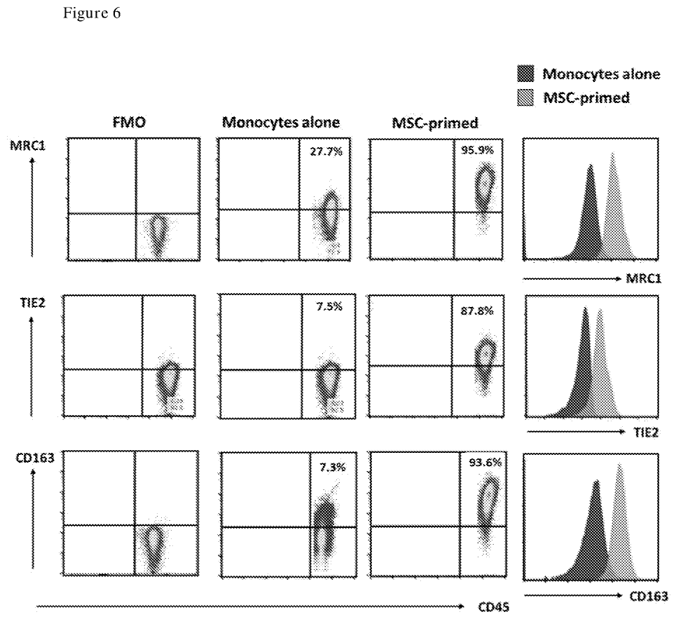

The phenotype of monocytes cultured alone those co-cultured with adMSCs, for a range of time points (3, 7, 14 and 21 days) at a 2:1 monocytes: adMSC ratio, was investigated using flow cytometry (n=6 samples per time point, see FIGS. 5 and 6 for example staining).

### Day 3

### Upregulated Markers

There was a significant increase in the percentage-positive expression of the ‘M2-like’ receptors CD206, TIE2 and CD163 in monocytes co-cultured with adMSCs compared with monocytes culture alone at day 3. There was also a significant increase in expression of the ‘M1-like’ receptor CD80 following co-culture with adMSCs.

### Downregulated Markers

There was a significant decrease in expression of the ‘M2-like’ receptor NRP1 when monocytes were co-cultured with adMSCs compared with monocytes alone. There was a significant decrease in HLA-DR expression following co-culture with ADMSCs.

There was no change in CD86 expression co-cultured monocytes compared with those cultured alone.

### Day 7

### Upregulated Markers

There was a significant increase in the percentage-positive expression of CD206, TIE2 and CD163 respectively in monocytes co-cultured with adMSCs compared with monocytes cultured alone at day 7. There was also a significant increase in the CD80 following co-culture with adMSCs.

There was no significant change in CD86, NRP1 or HLA-DR expression when monocytes were co-cultured with adMSCs compared with monocytes alone.

### Day 14

### Upregulated Markers

There was a significant increase in the percentage-positive expression CD206, TIE2 and CD163 in monocytes co-cultured with adMSCs compared with monocytes cultured alone by day 14. There was also a significant increase in CD80 expression following co-culture with ADMSCs. The expression of HLA-DR was also significantly increased.

### Downregulated Markers

There was a significant decrease in NRP1 expression when monocytes were co-cultured with adMSCs compared with monocytes alone. There was a significant decrease in CD86 expression following co-culture with ADMSCs by day 14.

### Day 21

### Upregulated Markers

There was a significant increase in the expression of CD206, TIE2 and CD163 respectively in monocytes co-cultured with adMSCs compared with monocytes culture alone at day 21. There was no difference in HLA-DR and NRP1 expression following co-culture with adMSCs. There was also a significant increase in CD80.

There was a no change in CD86 expression when monocytes were co-cultured with adMSCs compared with monocytes alone.

### Expression of Pro-Inflammatory CD38 Expression is Upregulated in MSC-Primed Monocytes

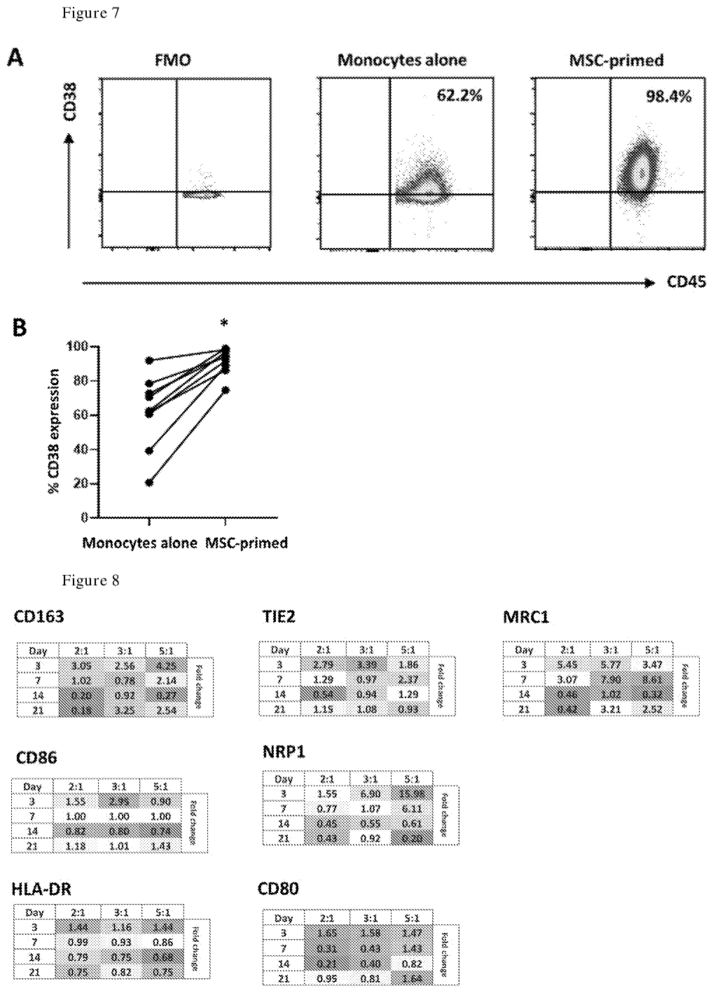

We assessed key ‘M1-like’ pro-inflammatory markers CD38 and CD80. The percentage of CD38 expressing monocytes cells significantly increases following adMSC priming at both 3 (62.2±7.1% vs 91.7±2.6%, P<0.001), n=9, FIG. 7) and 7 (54.7±6.4% vs 85.0±4.4, P<0.01, n=8) days. CD80 expression also significantly increased in co-culture at 3 days of adMSC priming.

FIG. 5 shows flow cytometric identification of monocytes. Monocytes alone in culture (A) or cultured with adMSCs (B) are identified according to CD45 expression following doublet and dead cell exclusion.

FIG. 6 shows example flow cytometric dot plots and histograms of increase in CD206, TIE2 and CD163 and CD86 following co-culture of monocytes with adMSCs

FIG. 7 shows increase in CD38 expression following priming of monocytes with adMSCs. (A) Example flow cytometric dot plot of Increase in CD38 expression at day 3 following priming of monocytes with adMSCs. (B) Overall, there is a significant increase in CD38 expression following priming of monocytes with adMSCs, *P<0.05

TABLE 4
Percentage expression of surface receptors following culture of monocytes alone or with adMSCs (MSC-Primed).

| | Day 3 | | | Day 7 | | | Day 14 | | | Day 21 | | |
|---|---|---|---|---|---|---|---|---|---|---|---|---|
| | (% expression) | | | (% expression) | | | (% expression) | | | (% expression) | | |
| | | MSC- | | | MSC- | | | MSC- | | | MSC- | |
| | Alone | Primed | P | Alone | Primed | P | Alone | Primed | P | Alone | Primed | P |
| CD206 | 3.5 ± 1.2 | 98.3 ± 10.7 | * | 6.2 ± 2.4 | 76.0 ± 6.2 | * | 25.2 ± 10.8 | 68.2 ± 10.9 | * | 25.9 ± 14.9 | 77.2 ± 5.5 | * |
| TIE2 | 5.7 ± 2.8 | 93.3 ± 5.8 | * | 2.4 ± 0.9 | 12.9 ± 2.2 | * | 46.9 ± 12.2 | 82.0 ± 5.7 | * | 39.5 ± 11.0 | 86.9 ± 11.5 | * |
| CD163 | 6.7 ± 2.1 | 77.7 ± 3.3 | * | 5.5 ± 1.5 | 33.8 ± 3.4 | * | 12.1 ± 4.3 | 24.7 ± 10.4 | * | 9.7 ± 3.2 | 23.2 ± 3.8 | * |
| CD86 | 94.3 ± 1.5 | 97.2 ± 0.9 | ns | 74.4 ± 2.0 | 97.9 ± 0.5 | ns | 27.8 ± 17.6 | 19.3 ± 13.2 | * | 55.6 ± 12.3 | 47.4 ± 15.2 | ns |
| CD80 | 70.7 ± 12.4 | 94.3 ± 1.5 | * | 32.2 ± 3.9 | 71.7 ± 2.2 | * | 2.2 ± 0.2 | 55.9 ± 14.3 | * | 2.4 ± 1.1 | 27.2 ± 7.4 | * |
| NRP1 | 50.6 ± 19.3 | 16.6 ± 7.0 | * | 7.5 ± 0.6 | 6.9 ± 1.1 | ns | 40.7 ± 10.1 | 4.9 ± 1.0 | * | 8.0 ± 0.1 | 6.3 ± .0.02 | ns |
| HLA-DR | 97.6 ± 0.9 | 77.7 ± 8.4 | * | 94.4 ± 2.0 | 77.4 ± 2.0 | ns | 75.6 ± 15.9 | 89.9 ± 3.7 | * | 58.5 ± 17.9 | 52.5 ± 17.9 | ns |

* P < 0.05

Optimisation of Cell Ratios for Monocyte Priming with adMSCs

The importance of monocyte: adMSC ratio on optimal monocyte priming was investigated (n=5 samples/ratio/time-point, FIG. 8). Upregulation of TIE2 and was greatest at a 3:1 ratio (7.6±3.3% vs 84.2±12.2%). The increase in CD206 expression was higher at 2:1 and 3:1 ratios (12.2±2.7% vs 92.3%±18.7% and 6.5±3.2% v 79±18.6% respectively) and CD163 expression most elevated at 5:1 (13.9%±5.4 vs 88.8%±12.9%).

By day 7 the expression of the three ‘M2-like’ markers, co-cultured with ADMSCs were significantly elevated (but still less than at day 3) at a 5:1 ratio (TIE2: 13.46%±1.14 vs 76.8%±14.18, CD206: 9.09%±0.89 vs 74.4%±9.89, CD163: 13.45%±2.34 vs 94.2%±18.7) showing a 4.2, 11.0 and 10.8-fold increase, respectively (P<0.0001 by Kruskal-Wallis test).

FIG. 8 shows heat maps showing overall optimisation of fold-change increases in monocyte cell surface expression (compared with 1:1 ratio for that time point)

### Validation of Optimised Monocyte Priming Experiments

The first set of experiments suggested that monocyte skewing when co-cultured with adMSCs was optimal from days 3 to 7 at a ratio of 3:1. Further experiments were carried out to confirm and validate the time point that produces the optimal monocyte phenotype (n=21 samples). Skewing of monocytes in co-culture was greater at day 3 compared to days 5 and 7 (CD206: 89.2% vs 62.1% vs 42.4%; TIE2: 75.3% vs 50.4% vs 32.3%; CD163: 78.3% vs 72.4% vs 28.9% respectively). The fold-change increase in expression of each of the three markers was highest after 3 day of culture compared with days 5 and 7 (CD206: 13.3 vs 4.1 vs 2.8-fold; TIE2: 16.8 vs 7.8 vs 4.3-fold; CD163: 7.0 vs 4.7 vs 2.8-fold respectively).

TABLE 5
Validation of monocyte priming experiments to compare days 3, 5 and 7.

| | Day 3 | | | Day 5 | | | Day 7 | | |
|---|---|---|---|---|---|---|---|---|---|
| | (% expression) | | | (% expression) | | | (% expression) | | |
| | Alone | MSC-primed | P | Alone | MSC-primed | P | Alone | MSC-primed | P |
| CD206 | 12.9 ± 2.2 | 89.2 ± 3.3 | * | 22.6 ± 3.7 | 62.1 ± 7.0 | * | 13.9 ± 4.2 | 32.4 ± 6.8 | * |
| TIE2 | 9.2 ± 1.5 | 75.3 ± 4.5 | * | 15.9% ± 7.0 | 50.4% ± 9.9 | * | 11.6 ± 2.7 | 32.2 ± 5.8 | * |
| CD163 | 23.6 ± 4.9 | 78.3 ± 3.1 | * | 30.8 ± 6.5 | 72.4 ± 4.7 | * | 15.9 ± 4.7 | 28.9 ± 7.2 | * |

* P < 0.05

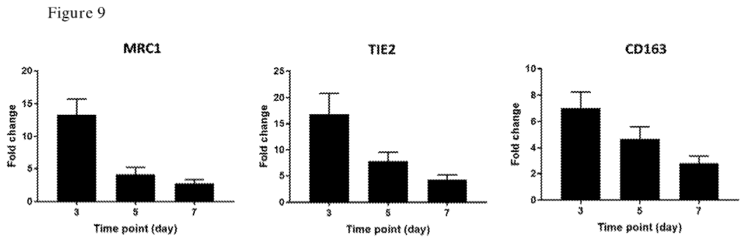

FIG. 9 shows that the fold-change increases in CD206, TIE2 and CD163 are greatest following 3 days of priming of monocytes with adMSCs

### Sequential Flow Cytometric Gating Confirms Day 3 is an Optimal Time-Point for the Engineering (‘Educating’ or ‘Priming’ or ‘Producing’) of “Triple Positive” Monocytes

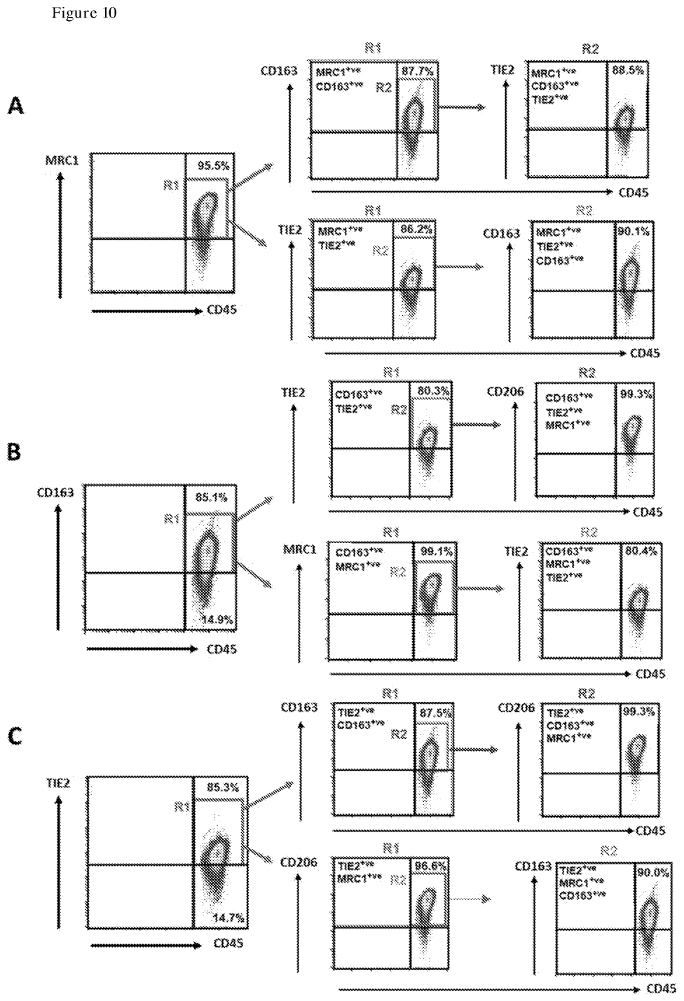

A gating strategy was used to determine the percentage of cells that are single, double and triple positive for the markers CD206, TIE2 and CD163 (FIG. 10). Following 3 days of priming with adMSCs, there was a significant increase in single receptor (CD206: 11.2±8.7% vs 91.6±13.8%; CD163: 18.4±11.8% vs 82.4±10.7%; TIE2: 9.1±7.1% vs 82.6±9.1%) and double receptor (CD206⁺/CD163⁺: 4.9±4.1% vs 80.7±13.6%; CD163±/TIE2±: 5.8±5.3% vs 73±9.5%; TIE2⁺/CD206⁺: 5.4±4.5% vs 78.1±12.9%) expression compared with monocytes cultured alone (P<0.0001 by Wilcoxon test, n=21 samples). Triple positive receptor expression of adMSC-primed monocytes (70.0±14.7%) was significantly higher than monocytes cultured alone (3.2±3.5%, P<0.0001, FIG. 11).

Following 7 days of priming with adMSCs, there was an increase in single receptor (CD206: 14.2±8.3% vs 35.3±17.5%; CD163: 10.0±15.8% vs 25.1±27.2%; TIE2: 11.5±11.9% vs 31%±24.7) and double receptor (CD206⁺/CD163⁺: 0.5±0.5% vs 22.9±17%; CD163⁺/TIE2⁺: 0.4±0.5% vs 24.8±22.2%; TIE2⁺/CD206⁺: 1.5%±2.8% vs 22.2%±16.2%) in adMSC-primed monocytes compared with monocytes cultured alone (P<0.0001 by Wilcoxon test, n=21). Triple positive receptor expression of adMSC-primed monocytes (16.3±15.3%) was significantly higher than monocytes cultured alone (0.09±0.1%, P<0.05, FIG. 11). However, the day 3 time point of adMSC-priming resulted in far greater triple positive cells (70±14.7%) compared with day 7 (3.2±3.5%).

FIG. 10 shows sequential gating strategy of monocyte markers. CD45+ monocytes are gated for their single-positive expression of CD206, CD163 and TIE2 based on FMO controls. Double positivity is then identified by gating this population (R1) for the other two markers (R2 and R3). The Ra and R3 double-positive populations are then gated for the third marker to determine the proportion of triple positive cells.

FIG. 11 shows Venn diagrams of single, double and triple percentage positivity of CD206, CD163 and TIE2 expression of monocytes following 3 and 7 days of culture with or without MSCs. The single, double and triple-positive staining is significantly greater following 3 days of ad-MSC priming compared with 7 days (n=21 samples, P<0.0001 by Wilcoxon test).

Proteome Profile of Patient Monocyte Following Co-Culture with MSCs

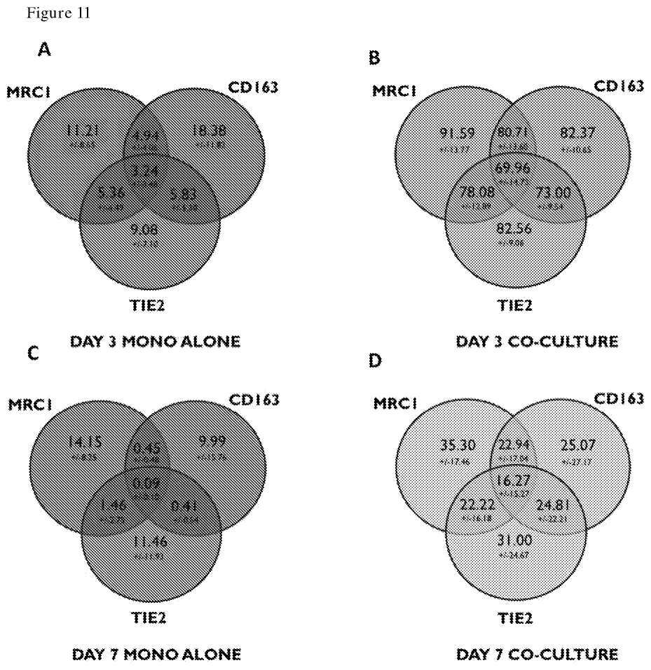

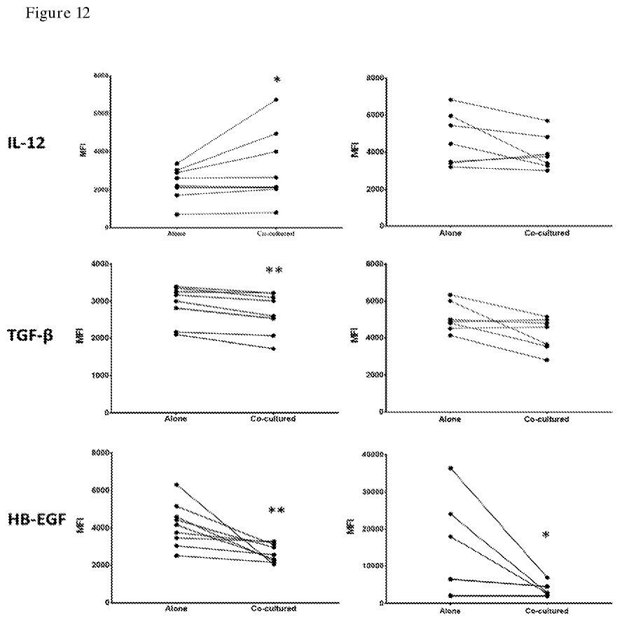

The monocytes co-cultured with adMSCs using our optimised technique do not show a specific known ‘M1’ or ‘M2’ phenotype. We determined the functional effect of co-culture by measuring proteins in the conditioned media of these cells. After 3 days of co-culture, HB-EGF and TGFβ were significantly reduced (P<0.005 for both, FIG. 12) and IL-12 was significantly increased (P<0.05). By day 7, there was no difference in TGFβ or IL-12 between monocytes co-cultured with adMSCS and those cultured alone, whereas HB-EGF was still significantly reduced (P<0.05).

FIG. 12: Changes in monocyte secretome following priming with adMSCs. Changes in IL12, TGFβ and HB-EGF are time dependent.

Priming (‘Educating’) of Monocytes from Patients with Critical Limb Ischaemia

The cells/populations of cells of the invention may be used as an autologous cell therapy. We thus aimed to ensure that our optimised method can be used to prime monocytes isolated from patients with critical limb ischaemia. Our optimised method results in a significantly higher fold-change at day 3 versus day 7 for TIE2 (16.1±5.3% VS 2.2±0.4% respectively, P<0.05), CD206 (7.2±2.6% vs 1.4±0.1%, P<0.005) and CD163 (5.3±1.3% vs 1.4±0.3%, P<0.05, FIG. 13).

FIG. 13: Priming of monocytes from patients with CLI. Significantly greater fold-change in expression of all three markers, TIE2, CD206 and CD163, after priming for 3 days compared with 7 days.

Similar to the culture of monocytes from control donors, the triple positive receptor expression of monocytes from patients with CLI was significantly higher than monocytes primed with adMSCs after 3 days (adMSC primed: 55.6±10.3% vs cultured alone: 0.3±0.1%), but not 7 days (0.3.6±0.2% vs 3.5±3.4%, FIG. 14).

FIG. 14: Venn diagrams of single, double and triple percentage positivity of CD206, CD163 and TIE2 expression of monocytes from CLI patients following 3 and 7 days of culture with or without MSCs. The single, double and triple-positive staining is greater following 3 days of adMSC-priming, but not 7 days (n=3 CLI samples).

In Vitro and In Vivo Function of Monocytes Primed with adMSCs

### MSC-Primed Monocytes Induce Smooth Muscle Cell Proliferation

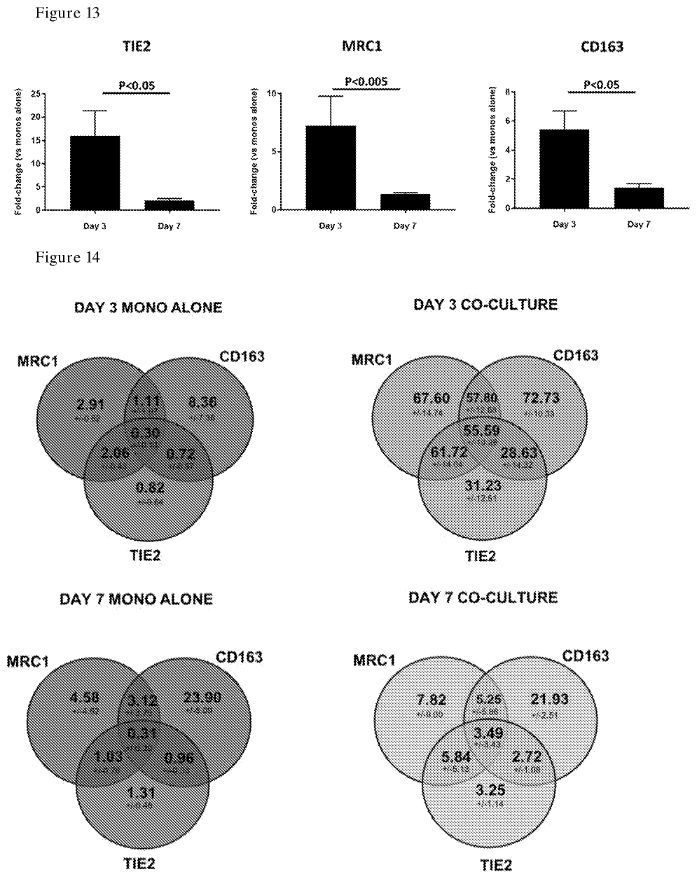

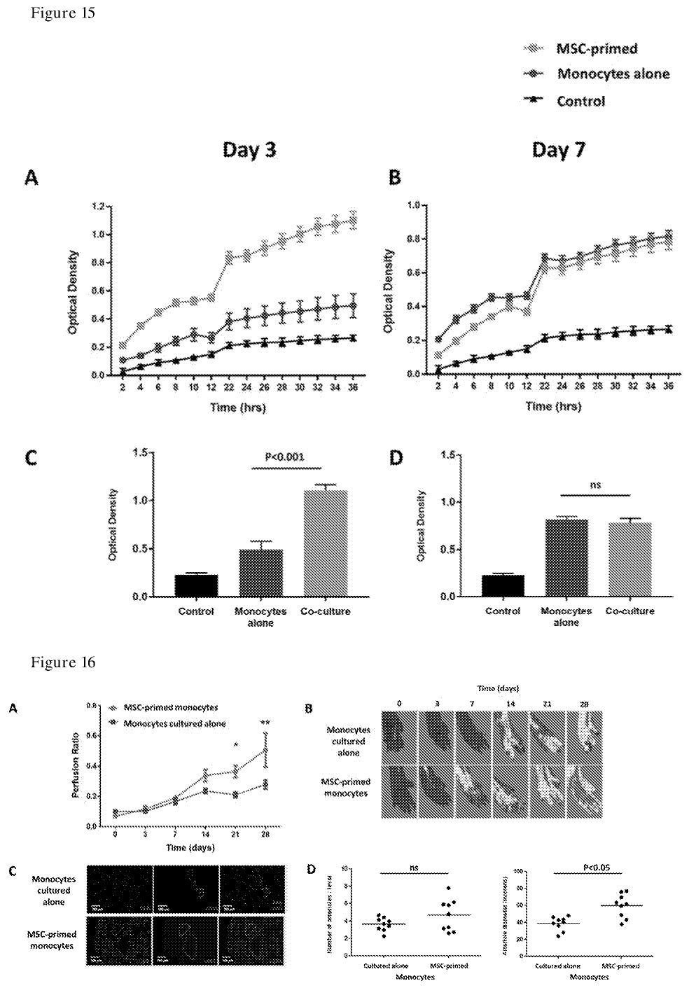

We compared the in vitro function our adMSC-primed monocytes with non-primed monocytes using our optimised ratio (3:1) following 3 and 7 days of culture. There was a significant increase in SMC proliferation using conditioned medium from monocytes primed with adMSCs for 3 days compared with both monocytes plated alone and control media (two-way repeated measures ANOVA, P<0.001, FIG. 15). On the other hand, there was no significant difference in the in vitro between primed and non-primed monocytes following 7 days of culture.

### MSC-Primed Monocytes Regulate In Vivo Post-Ischaemic Neovascularisation

We compared the in vivo function our adMSC-primed monocytes with non-primed monocytes using our optimised ratio (3:1) and time point (3 days of priming) by delivering these cells into the ischaemic murine hindlimb.

Suitably 1×10\hat{ }6 cells are delivered. Suitably said 1×10\hat{ }6 cells comprise at least 50% macrophage or monocyte cells, characterised in that at least 50% of said macrophage or monocyte cells express each of the markers: MRC1; TIE2; and CD163. Suitably said cells are delivered in saline buffer. Suitably said cells are delivered by intramuscular injection. Suitably a single dose of cells is administered.

We found a significant increase in revascularisation seen with laser Doppler imaging of ischaemic paws following delivery of adMSC-primed monocytes compared with monocytes alone (P<0.01 by repeated measures two-way ANOVA, FIG. 16). Immunohistochemical analysis of injected muscle showed that the mechanism of increased revascularisation by these adMSC-primed monocytes was as a result of greater arteriogenesis within the target tissue by way of an increase in the number and diameter of arterioles

FIG. 15: Smooth muscle cell proliferation potential of adMSC-primed monocytes and monocytes culture alone. Example graphs of XTT measurements showing greater smooth muscle cell (SMC) proliferation following culture with conditioned media (CM) from adMSC-primed monocytes compared with non-primed monocytes after 3 (A) but not 7 (B) days of priming. (C) Overall, after day 3, there is a significant increase in SMC proliferation in response to CM from adMSC-primed monocytes compared with CM from monocytes cultured alone. This is not seen following 7 days of culture, where there is no difference in SMC proliferation from adMSC-primed monocytes compared with monocytes alone (D). n=11 separate monocyte samples, each performed in duplicate).

FIG. 16 shows revascularisation of adMSC-primed monocytes and monocytes culture alone following delivery into the ischaemic hindlimb. (A) Delivery of adMSC-primed monocytes into the ischaemic hindlimb results in significantly greater revascularisation compared with monocytes cultured alone by day 21 and 28 (P<0.01 by two-way repeated measures ANOVA, *P<0.05, **P<0.01 by post-hoc Bonferroni testing, n=5 mice/group). (B) Example laser Doppler images of a mouse treated with monocytes cultured alone compared with adMSC-primed monocytes, showing greater paw perfusion by day 21 and 28. (C) Example immunohistochemistry showing greater arteriole (SMA staining, red) size in muscle from mice following treatment with adMSC-primed monocytes compared with monocytes cultured alone. (D) Overall, there is significantly greater arteriogenesis (number of arterioles) following treatment of limbs with adMSC-primed monocytes compared with monocytes cultured alone (n=9 mice/group).

FIG. 17 shows the phenotype of monocytes following co-encapsulation with MSCs. These monocytes upregulate their expression of TIE2. MRC1 and CD163.

FIG. 18 shows that the MSC-primed monocytes exhibit significantly downregulated expression of MMP-9 compared with monocytes cultured alone.

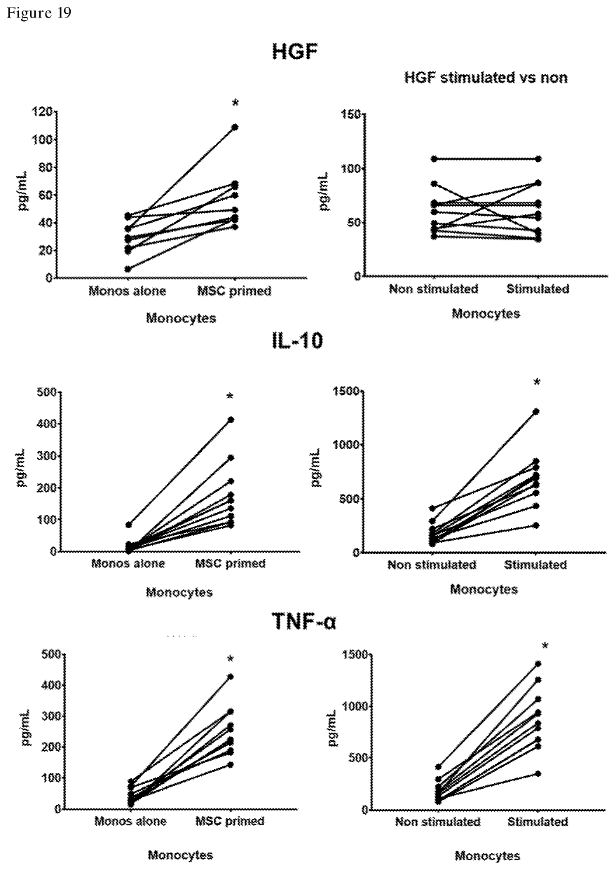

FIG. 19 shows that MSC primed monocytes express significantly higher levels of HGF, IL-10 and TNF-α. These proteins are known to have marked pro-angio/arteriogenic and anti-fibrotic activity. These cells are also able to further upregulate the expression of IL-10 and TNF-α when exposed to an inflammatory stimulus such as lipopolysaccharide (LPS). Furthermore, their primed state is not reversed by exposure to LPS as their expression of HGF is not reduced.

FIG. 21 shows Picrosirius red staining of murine adductor muscle following delivery of monocytes cultured alone (top) and MSC-primed monocytes. The product protects against fibrosis following tissue injury and ischaemia. Representative images taken using polarised light microscopy from 8 mice in each group. White arrows show areas of collagen deposition (green and orange/yellow fibres) which are reduced in mice treated with MSC-primed monocytes. Overall, there is a significant reduction in fibrosis following treatment with MSC-primed monocytes compared with whole population monocytes (mean 58%±4SEM; *P<0.005).

### Example 9: Safety Profile

Here we present mammalian (murine) safety assessment data.

In this experiment, 6 mice were injected intravenously via tail vein with 1×10⁶ MSC-primed monocytes (cells of the invention) into nude, athymic mice (n=6).

Survival was 100% at 6 weeks.

We refer to FIG. 23.

This demonstrates expected safety of these cells for human use.

### Example 10 Clinical Manufacture of Cells

The initial donor product (starting cells) can either be whole blood or leukapheresis product. For whole blood, red cell volume reduction using the “WB Step 1” cells wash programme on the Lovo™ Med device (Fresenius Kabi, Three Corporate Drive Lake Zurich, IL 60047, U.S.A.) is required first prior to proceeding to monocyte labelling, whereas this is not needed for leukapheresis product. Monocytes/macrophage are then isolated by labelling the cell suspension with anti-CD14 magnetic beads and passing it though a magnetic column CliniMACS Plus cell processor (Catalogue Number 151-01 Miltenyi Biotec, address ibid.) to enrich for CD14+ cells.

‘Priming’ (Co-Culture with MSCs)

The first step is to generate a bank of MSC vials that can subsequently be thawed for manufacture of each clinical batch. The MSCs are sourced from RoosterBio Inc, 5295 Westview Drive, Suite 275, Frederick, MD 21703 (RoosterVial™-hBM-20M-XF, MSC-CC040) and manufactured and expanded according to the manufacture's guidelines prior to storage (in vials containing either 10⁷ or 10⁸ cells so that both formats are available in different amounts depending on the scale of the ensuing co-culture manufacture.)

The next step is to culture these monocytes/macrophage cells on a confluent layer of MSCs for a period of usually up to 7 days, more suitably up to 5 days.

To generate this confluent layer, one vial of 10⁷ MSCs per CellSTACK, Corning Cat. No. 3330) is needed. Therefore, if a CellSTACK 10 (Corning Cat. No. 3312) is used for larger scale manufacture, a vial of 10⁸ MSCs needs to be thawed. MSCs are thawed 72 hrs prior to co-culture with the monocytes/macrophages and cultured with RoosterNourish media in the appropriate sized CellSTACK for 72 hours This results in a confluent (over 80%) later of MSCs within the CellSTACK(s). 72 hours later, the monocytes/macrophages are added to the MSCs, at which point the media is changed to X-vivo 10 media (Lonza)

Media changes are not needed. However, the culture period may be extended to 7 days (with a media change at day 5) if TIE2 levels are not increased after 5 days of culture. Here “increased” means expression raised to a level higher than the starting cells.

Suitably “increased” means expression raised to a level higher than the starting cells, in at least 50% of the monocytes/macrophage present.

The co-cultured cells are then lifted, re-incubated with anti-CD14 beads and processed through the magnet again to separate the monocytes/macrophage from the MSCs. The resulting monocyte-enriched population of cells can be analysed for purity by way of CD45 expression as MSCs do not express this marker.

The cells may then be formulated in Plasma-Lyte 148 supplemented with 5% v/v human serum albumin and 10% v/v DMSO, and optionally frozen in a controlled rate freezer.

### Example 11—Exemplary Method of Manufacture

### Step 1

The initial donor product can either be whole blood or leukapheresis product. For whole blood, 350-485 mLs of blood is collected and the “WB Step 1” cells wash programme on the Lovo Med device (Fresenius Kabi) used to reduce the volume to approximately 150 mLs. The resulting red-cell reduced suspension is collecting into a transfer bag. For leukaphereis product, the cell suspension does not require a reduction in volume.

### Step 2: Enrichment of CD14+ Monocytes

If the initial donor product is whole blood, 1/100 of the red cell reduced volume (approximately 1.4-1.5 ml) of CliniMACS CD14 Reagent (Miltenyti Biotec, 170-076-705) is transferred into the volume-reduced cell suspension. If the initial product is leukapheresis, 5 mL of the CD14 Reagent is transferred. The Reagent is incubated between 2 and 10° C. for 20 minutes with continuous, gentle agitation. Following incubation, the cells are processed through the CliniMACS Plus cell processor (Miltenyi Biotec) following the manufacturer's written instructions, using the pre-set program for enrichment (Enrichment 3.2). The CD14+ target cells are collected in the cell collection tube. On completion of the automatic program the collection tube containing the CD14+ enriched monocytes is centrifuged at 750×g for 10 minutes. The cells are then resuspended in 40 mL of X-Vivo 10 Medium (Lonza).

### Step 3: Co-Culture

For whole blood initial donor: if the cell number is less than 1×10⁸ total monocytes, cells are seeded into one CellSTACK (Corning, 3330). If more than 1×10⁸ monocytes are isolated from the patient's blood, then they are seeded into two CellSTACKs (Corning. 3310).

For leukapheresis donor: A maximum of 1×10⁹ monocytes are seeded onto a CellSTACK-10 (Corning, 3312). Cells are cultured on a confluent (80-90% confluency) layer of MSCs (RoosterBio) for a period of usually up to 5 days. Media changes are not needed.

The culture period may be extended to 7 days (with a media change at day 5) if the percentage of monocytes expressing TIE2 is not greater than 50% after 5 days of culture.

### Step 4: Product Harvest

To lift the adherent cells from the CellSTACKs, the media is removed and 20 mL of pre-warmed TrypLE (ThermoFisher, A1285901) added to each CellSTACK layer for 12 minutes in a 37° C. incubator, agitating every 3-4 minutes. The TrypeLE is quenched with CliniMACS PBS/EDTA Buffer+0.5% HAS. The final volume is brought to 180 ml (using the Lovo for volume reduction as above if the original donor product was leukapheresis). The monocyte-MSC suspension is re-incubated with 1.8 ml CliniMACS CD14 Reagent (1/100). Mnocytes can then be separated from the MSCs using either the CliniMACS Plus enrichment programme or magnetic separation LS columns and a QuadroMACS Separator (Miltenyi Biotec). The LS columns are required when the initial product is whole blood as the cell numbers are lower and the CliniMACS Plus is not adequate to ensure high yield and purity in this situation. Retained cells are resuspended in a total volume of 55 mL PlasmaLyte-148+10% HSA. The resulting monocyte-enriched population of cells can be analysed for purity by determining the proportion of cells that express CD45 expression as MSCs do not express this marker. The cells are formulated in Plasma-Lyte 148 supplemented with 5% v/v human serum albumin and 10% v/v DMSO, and frozen in a controlled rate freezer.

### Example 12: Reproducibility of Manufacture

We present data showing reproducibility of the method (manufacture).

We refer to FIG. 4 which shows bar charts demonstrating reproducibility data of expression of CD206, CD163 and TIE2 from 3 separate technicians generating cells of the invention (MSC-primed monocytes) as in the above examples.

Technician A: n=10 samples, B: n=6 samples, C: n=5 samples.

No significant difference was seen between the technicians for all 3 markers. Thus the methods disclosed are reproducible.

### Example 13—25F9

We refer to FIG. 25.

There is higher expression of 25F9 expression in MSC-primed monocytes at every timepoint compared with monocytes cultured alone. Monocytes cultured alone do not increase their expression of 25F9 before day 7, whereas the MSC-primed monocytes express increasing levels of 25F9 in response to the culture conditions.

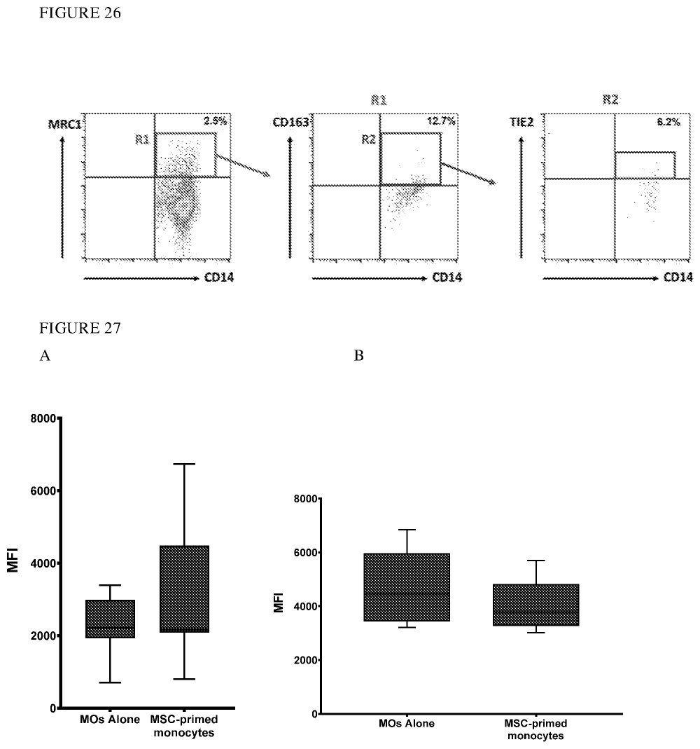

We refer to FIG. 26.

Flow cytometric dot plot showing sequential gating of circulating monocytes in the blood. In this example, 10,365 monocytes were analysed in this blood sample of which 8/10,365 cells are triple positive (0.08%).

Blood monocytes (primary monocytes) do not express the marker 25F9, whereas the MSC-primed cells (cells of the invention) significantly upregulate this marker by day 3.

### Example 14—Sustained Expression/Effective Timings of Induction

Prior art expression levels of various markers are markedly lower than cells of the invention, and are not clinically useful.

We also demonstrate that these levels drop further with prolonged culture, emphasising the valuable contribution of the timings in the methods disclosed herein.

It must be noted that in prior art methods, most or all of the cells are cultured for more than 7 days, as in prior art methods the monocytes are cultured alone for a few days first (which produces differentiation into macrophages), before being co-cultured with MSCs. In contrast the present invention teaches the direct co-culture of monocytes (primary monocytes) with MSCs from day 0.

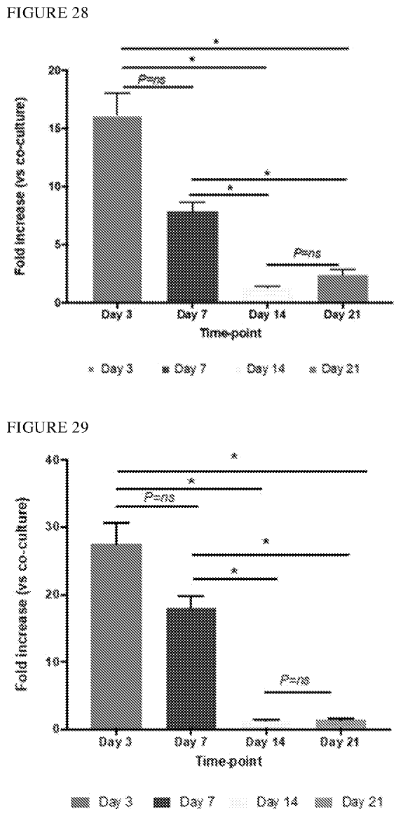

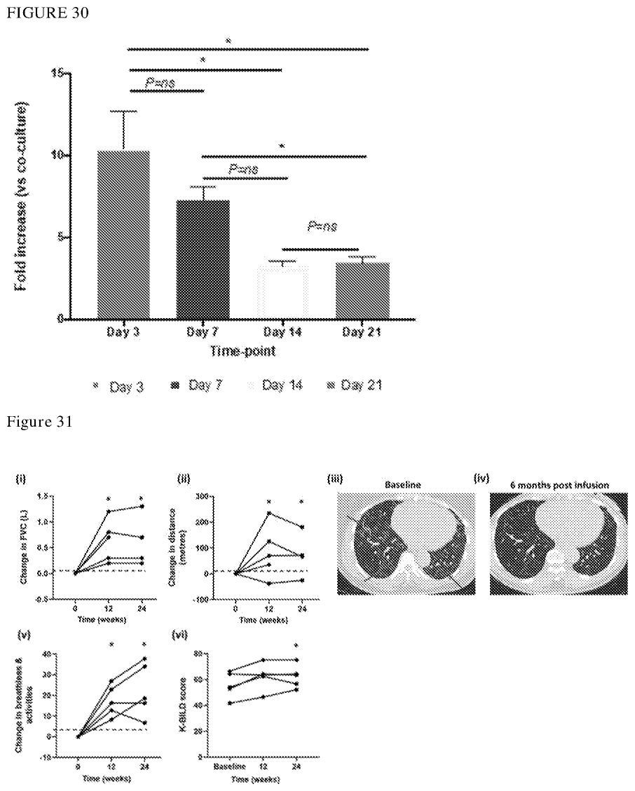

We refer to FIG. 28, FIG. 29 and FIG. 30 showing this i.e. showing that the levels of the three key markers CD202B (TIE2), CD163 and CD206 (MRC1) drop past day 7, i.e. levels are excellent within the timings taught herein, and levels drop if cultured outside the timings taught herein.

### Example 15—Demonstration in Humans

The MONACO Cell Therapy Study: Monocytes as an Anti-fibrotic Treatment After COVID-19 (NCT0480508) is a Phase 1, open-label clinical trial to assess the safety and tolerability of a single intravenous dose of our product (i.e. cells of the invention) (i.e. MSC-primed monocytes) in patients with fibrotic lung disease following COVID-19 infection. 5 patients were recruited into this study.

We found a significant median absolute increase in FVC 12 and 24 weeks respectively compared with baseline (FIG. 31 (i), *P<0.05). There was also significant increase in walking distance (FIG. 31 (ii), *P<0.05), improvement in breathlessness and K-BILD score at 24 weeks (FIG. 31 (v) and FIG. 31 (vi)).

FIG. 31 (iii) and FIG. 31 (iv) show a patient with improvement in areas of lung fibrosis (see arrows) (red arrows as filed)) after infusion of the MSC-primed monocytes.

Thus beneficial technical effects of the invention are demonstrated in human subjects.

1. A population of cells,
wherein said population of cells comprises at least 50% myeloid cells such as macrophage or monocyte cells,
characterised in that at least 50% of said myeloid cells such as macrophage or monocyte cells express each of the markers:
MRC1;
TIE2; and
CD163.

2. The population of cells according to claim 1, wherein said population of cells comprises at least 70% myeloid cells such as macrophage or monocyte cells.

3. The population of cells according to claim 1, wherein said population of cells comprises at least 80% myeloid cells such as macrophage or monocyte cells.

4. The population of cells according to claim 1, wherein at least 60% of said myeloid cells such as macrophage or monocyte cells express each of the markers:
MRC1;
TIE2; and
CD163.

5. The population of cells according to claim 1, wherein said myeloid cells such as macrophage or monocyte cells express CD14 and/or CD45.

6. The population of cells according to claim 1, wherein said myeloid cells such as macrophage or monocyte cells express TNFalpha (TNFa) and/or IL-12.

7. The population of cells according to claim 1, comprising a 3:1 ratio of myeloid cells such as monocyte/macrophage cells:MSCs.

8. A method comprising
(a) providing a monocyte from a subject
(b) providing a MSC
(c) culturing said monocyte with said MSC,
wherein step (c) comprises contacting said monocyte with said MSC to produce a cell mixture, and culturing said cell mixture.

9. The method according to claim 8, wherein the ratio of monocytes:MSCs in step (c) is 3:1.

10. The method according to claim 8, wherein the cells are cultured for about 3 to 7 days.

11. The method according to claim 8, wherein the cells are cultured for about 3 days.

12. The method according to claim 8, wherein the cells are cultured in a medium, and wherein said medium is changed every 5 days.

13. A method of treating peripheral vascular disease, suitably chronic limb threatening ischaemia, comprising administering the population of cells according to claim 1.

14. A method of treating fibrosis, comprising administering the population of cells according to claim 1.

15. A method of treating ischaemic stroke, comprising administering the population of cells according to claim 1.

16. A method of treating a mammalian subject comprising administering the population of cells according to claim 1 to said subject.

17. The method according to claim 16, comprising administering a dose of 100 million to 200 million said cells.

18. The method according to claim 16, wherein said method induces angiogenesis in a subject.

19. The method according to claim 8, wherein the cells are cultured for about 3 to 5 days.
